SEQUENCE LISTING (13 sequences)

SEQ ID NO: 1 - DNA, 1697 bp, respiratory syncytial virus
atggagttgctaatcctcaaagcaaatgcaattaccacaatcctcactgcagtcacatttgttttgcttctggtcaaaacatcactgaagaattttatcaatcaacatgcagtgcagtagcaaaggctatcttagtgctctgagaactggttggtataccagtgttataactatagattaagtaatatcaaggaaaataagtgtaatggaacagatgctaaggtaaaattgataaacaagaattagataaatataaaaatgctgtaacagaattgcagttgctcatgcaaagcacccagcaacaaacaatcgagccagaagagaactaccaaggtttatgaattatacactcaaaatgccaaaaaaaccaatgtaacattaagcaagaaaaggaaaagaagatttcttggtttttgttaggtgttggatctgcaatcgccagtggcgttgctgtatctaaggtcctgcacctgaaggggaagtgaacaagatcaaaagtgctctactatccacaaacaaggctgtagtcagttatcaaatggagttagtgtcttaaccagcaaagtgttagacctcaaaaactatatagaaaacaattgttacctattgtgaacaagcaaagctgcagcatatcaaatatagcaactgtatagagttccaacaaaagaacaacagactactagagattaccagggaatttagtgttaagcaggtgtaactacacctgtaagcacttacatgttaactaatagtgaattattgtcattatcaatgatatgcctataacaaatgatcagaaaaagttaatgtccaacaatgttcaaatgttagacagcaaagttactctatcatgtccataataaaagaggaagtcttagcatatgtgtacaattaccactatatggtgttatagatacaccctgttggaaactacacacatccccctatgtacaaccaacacaaaagaagggtccaacatctgtttaacaagaactgacagaggtggtactgtgacaatgcaggatcagtatctttcttcccacaagctgaaacatgtaaagtcaatcaaatcgagtattttgtgacacaatgaacagtttaacattaccaagtgaagtaaactctgcaatgttgacatattcaaccccaaatatgattgtaaaattatgacttcaaaaacgatgtaagcagctccgttatcacatctctaggagccattgtgtcatgctatggcaaaacaaatgtacagcatccaataaaaatcgtggaatcataaagacattttctaacgggtgcgatatgtatcaaataaaggggtggacactgtgtctgtaggtaacacattatattatgtaaaaagcaagaaggtaaaagtctctatgtaaaaggtgaaccaataataaatttctatgacccttagtattcccctctgatgaatttgatgcatcaatatctcaagtcaacgagaagattaacagagcctagcatttattcgtaaatccgatgaattattacataatgtaaatgctggtaatccaccataaatatcatgataactactataattatagtgattatagtaatattgttatcttaattgctgttggactgctcttatactgtaaggccagaagcacaccagtcacactaagaaagatcaactgagtggtataaataatattgcatttagtaactaa

SEQ ID NO: 2 - protein, 574 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIATVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 3 - DNA, 897 bp, respiratory syncytial virus
atgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtag

SEQ ID NO: 4 - protein, 298 aa, respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMETFHSTSSEGNLSPSQVSTTSEHPSQPSSPPNTTRQ

SEQ ID NO: 5 - protein, 14 aa, Artificial Sequence
  synthetic linker peptide
SGGSGGSGGSGGSG

SEQ ID NO: 6 - protein, 14 aa, Artificial Sequence
  synthetic linker peptide
SGGSGGSGTNVTLS

SEQ ID NO: 7 - protein, 16 aa, Artificial Sequence
  synthetic linker peptide
ELPRFMNYTLNNTKNT

SEQ ID NO: 8 - protein, 15 aa, Artificial Sequence
  synthetic linker peptide
QYTLNNTKNTNVTLS

SEQ ID NO: 9 - DNA, 2268 bp, Artificial Sequence
  recombinant chimeric FG polypeptide
atggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcctggtcacactaacaactgcaatcatacaagatgcaacaagccagatcaagaacacaaccccaacatacctcacccagaatccccagcttggaatcagcttctccaatctgtctgaaactacatcacaaaccaccaccatactagcttcaacaacaccaagtgtcaagtcaaccctgcaatccacaacagtcaagaccaaaaacacaacaacaaccaaaatacaacccagcaagcccaccacaaaacaacgccaaaacaaaccaccaaacaaacccaataatgattttcactttgaagtgttcaactttgtaccttgcagcatatgcagcaacaatccaacctgctgggctatctgtaaaagaataccaaacaaaaaacctggaaagaaaaccaccaccaagcccacaaaaaaaccaaccatcaagacaaccaaaaaagatctcaaacctcaaaccacaaaaccaaaggaagtacctaccaccaagcccacagaaaagccaaccatcaacaccaccaaaacaaacatcagaactacactgctcaccaacaataccacaggaaatccagaacacacaagtcaaaagggaaccctccactcaacctcctccgatggcaatccaagcccttcacaagtctatacaacatccgagtacctatcacaacctccatctccatccaacacaacaaaccag

SEQ ID NO: 10 - DNA, 1828 bp, Artificial Sequence
  recombinant polynucleotide encoding a chimeric FG polypeptide
aagcttgccaccatggagctgctgatcctgaaaaccaacgccatcaccgccatcctggccgccgtgaccctgtgcttcgcctcctcccagaacatcaccgaagagttttaccagtccacctgctccgccgtgtccaagggctacctgtccgccctgcggaccggctggtacacctccgtgatcaccatcgagctgtccaacatcaaagaaaacaagtgcaacggcaccgacgccaaggtcaagctgatcaagcaggaactggacaagtacaagagcgccgtgacagaactccagctcctgatgcagtccacccctgccaccaacaacaagaagtccggcggcagcggcggctctggcggctccggcggatctggcaagaagttcctgggcttcctgctgggcgctggctccgccatcgcctccggcaccgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccaaaaaaagctgatgtccaacaacgtgcagatcgtgcggcagcagtcctacagcatcatgagcatcatcaaggaagaagtcctggcctacgtcgtgcagctgcctctgtacggcgtgatcgacaccccttgctggaagctgcacacctcccccctgtgcaccaccaacaccaaggaaggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggaaggcaagagcctgtacgtgaagggcgagcctatcatcaacttctacgaccctctggtgttcccttccgacgagttcgacgcctccatcagccaggtcaacgagaagatcaaccagtccctggccttcatccggaagtccgacgagctgctgcacaacgtgaacgctggcaagtctaccaccaacatcatggtgaccaagcagcggcagaacaagcctcctaacaagcccaacaacgacttccacttcgaggtgttcaacttcgtgccttgctccatctgctccaacaaccctacctgctgggccatctgcaagagaatccccaacaagaagccaggcaagaaaaccaccaccaagcctaccaagaagcctaccttcaagaccaccaagaaggaccacaagcctcagaccacaaagcctaaggaagtgccaaccaccaagcaccaccaccatcaccactgataatcta

SEQ ID NO: 11 - protein, 602 aa, Artificial Sequence
  recombinant polynucleotide FG polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKKSGGSGGSGGSGGSGKKFLGFLLGAGSAIASGTAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMVTKQRQNKPPNKPNNDFHFEVFNFVPCSICSNNPTCWAICKRIPNKKPGKKTTTKPTKKPTFKTTKKDHKPQTTKPKEVPTTKHHHHHH

SEQ ID NO: 12 - DNA, 1849 bp, Artificial Sequence
  recombinant polynucleotide encoding a chimeric FG polypeptide
aagcttgccaccatggagctgctgatcctcaagaccaacgccatcaccgccatcctggccgccgtgaccctgtgcttcgcctcctcccagaacatcaccgaagagttctaccagtccacctgctccgccgtgtccaagggctacctgtccgccctgcggaccggctggtacacctccgtgatcaccatcgagctgtccaacatcaaagaaaacaagtgcaacggcaccgacgccaaggtcaagctgatcaagcaggaactggacaagtacaagagcgccgtgaccgaactccagctgctgatgcagtccacccctgccaccaacaacaagaaagaactgcctcggttcatgaactacaccctgaacaacaccaagaacaccaacgtgaccctgagcaagaagttcctgggcttcctgctgggcgctggctccgccatcgcctccggcaccgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgacaaactccgagctgctctccctgatcaacgacatgcctatcaccaacgaccaaaaaaagctgatgtccaacaacgtgcagatcgtgcggcagcagtcctacagcatcatgagcatcatcaaggaagaggtcctggcctacgtggtgcagctgcctctgtacggcgtgatcgacaccccttgctggaagctgcacacctcccccctgtgcaccaccaacaccaaggaaggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggaaggcaagagcctgtacgtgaagggcgagcctatcatcaacttctacgaccctctggtgttcccttccgacgagttcgacgcctccatcagccaggtcaacgagaagatcaaccagtccctggccttcatccggaagtccgacgagctgctgcacaacgtgaacgctggcaagtctaccaccaacatcatggtgaccaagcagcggcagaacaagcctcctaacaagcccaacaacgacttccacttcgaggtgttcaacttcgtgccttgctccatctgctccaacaaccctacctgctgggccatctgcaagagaatccccaacaagaagcctggcaagaaaaccaccaccaagcctaccaagaagcctaccttcaagaccaccaagaaggaccacaagcctcagaccacaaagcctaaggaagtgccaaccaccaagcaccaccaccatcaccactgataatcta

SEQ ID NO: 13 - protein, 609 aa, Artificial Sequence
  recombinant chimeric FG polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKKELPRFMNYTLNNTKNTNVTLSKKFLGFLLGAGSAIASGTAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMVTKQRQNKPPNKPNNDFHFEVFNFVPCSICSNNPTCWAICKRIPNKKPGKKTTTKPTKKPTFKTTKKDHKPQTTKPKEVPTTKHHHHHH

(19) United States
(12) Patent Application Publication
(10) Pub. No.: US 2011/0177117 A1
(43) Pub. Date: Jul. 21, 2011

(54) CHIMERIC RESPIRATORY SYNCYTIAL VIRUS POLYPEPTIDE ANTIGENS

(76) Inventors: Normand Blais, Laval (CA); Patrick Rheault, Laval (CA)

(21) Appl. No.: 13/054,651

(22) PCT Filed: Jul. 17, 2009
(86) PCT No.: PCT/CA2009/001021

§ 371 Date: Jan. 18, 2011

Related U.S. Application Data
(60) Provisional application No. 61/081,888, filed on Jul. 18, 2008.

(51) Int. Cl. A61K 39/155; C07K 14/135; A61K 39/295; C07H 21/00; C12N 5/10; C12N 1/00; A61P 31/14; A61P 37/04

(52) U.S. Cl. 424/201.1; 530/350; 424/211.1; 424/202.1; 536/23.72; 435/325; 435/243; 435/419

(57) ABSTRACT

Chimeric respiratory syncytial virus (RSV) polypeptide antigens are provided. The disclosed polypeptides include a first amino acid sequence comprising an F2 domain uncleavably joined to an F1 domain of a Respiratory Syncytial Virus (RSV) Fusion (F) protein polypeptide; and a second amino acid sequence comprising a portion of an RSV Attachment (G) protein polypeptide comprising an immunologically dominant epitope. The disclosure also provides nucleic acids that encode, and pharmaceutical compositions that contain, the chimeric RSV polypeptides, as well as methods for their production and use.

### CROSS-REFERENCE TO RELATED APPLICATION

This application claims benefit of the earlier filing dates of U.S. provisional application No. 61/081,888, filed 18 Jul. 2008, the disclosure of which is incorporated herein by reference.

### COPYRIGHT NOTIFICATION PURSUANT TO 37 C.F.R. §1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD

This disclosure concerns the field of immunology. More particularly, this disclosure relates to compositions and methods for eliciting an immune response specific for Respiratory Syncytial Virus (RSV).

### BACKGROUND

Human Respiratory Syncytial Virus (RSV) is the most common worldwide cause of lower respiratory tract infections (LRI) in infants less than 6 months of age and premature babies less than or equal to 35 weeks of gestation. The RSV disease spectrum includes a wide array of respiratory symptoms from rhinitis and otitis to pneumonia and bronchiolitis, the latter two diseases being associated with considerable morbidity and mortality. Humans are the only known reservoir for RSV. Spread of the virus from contaminated nasal secretions occurs via large respiratory droplets, so close contact with an infected individual or contaminated surface is required for transmission. RSV can persist for several hours on toys or other objects, which explains the high rate of nosocomial RSV infections, particularly in paediatric wards.

The global annual infection and mortality figures for RSV are estimated to be 64 million and 160,000 respectively. In the U.S. alone RSV is estimated to be responsible for 18,000 to 75,000 hospitalizations and 90 to 1900 deaths annually. In temperate climates, RSV is well documented as a cause of yearly winter epidemics of acute LRI, including bronchiolitis and pneumonia. In the USA, nearly all children have been infected with RSV by two years of age. The incidence rate of RSV-associated LRI in otherwise healthy children was calculated as 37 per 1000 child-year in the first two years of life (45 per 1000 child-year in infants less than 6 months old) and the risk of hospitalization as 6 per 1000 child-years (11 per 1000 child-years in the first six months of life). Incidence is higher in children with cardio-pulmonary disease and in those born prematurely, who constitute almost half of RSV-related hospital admissions in the USA. Children who experience a more severe LRI caused by RSV later have an increased incidence of childhood asthma. The costs of caring for children with severe LRI and their sequelae are substantial, and RSV is also increasingly recognized as a important cause of morbidity from influenza-like illness in the elderly, highlighting the need for a safe and effective vaccine capable of protecting against RSV-induced disease.

### SUMMARY

This disclosure concerns chimeric respiratory syncytial virus (RSV) antigens. The chimeric RSV antigens include, in an N-terminal to C-terminal direction: a first amino acid sequence comprising an F2 domain uncleavably joined to an F1 domain of a Respiratory Syncytial Virus (RSV) Fusion (F) protein polypeptide; and a second amino acid sequence comprising a portion of an RSV Attachment (G) protein polypeptide comprising an immunologically dominant epitope. The disclosed antigens elicit an immune response when administered to a subject, and can be used to treat and/or prevent the symptoms of RSV infection. Also disclosed are nucleic acids that encode the chimeric antigens, immunogenic compositions that contain the chimeric antigens, and methods for producing and using the chimeric antigens.

### BRIEF DESCRIPTION OF THE DRAWINGS

FIG. 1 is a schematic representation of the modification made with respect to the prototypical FG (FG Rix) to produce FG V1-1 and FG V2-1. Numerals 1 and 2 indicate the position of an introduced linker and the G protein fragment, respectively.

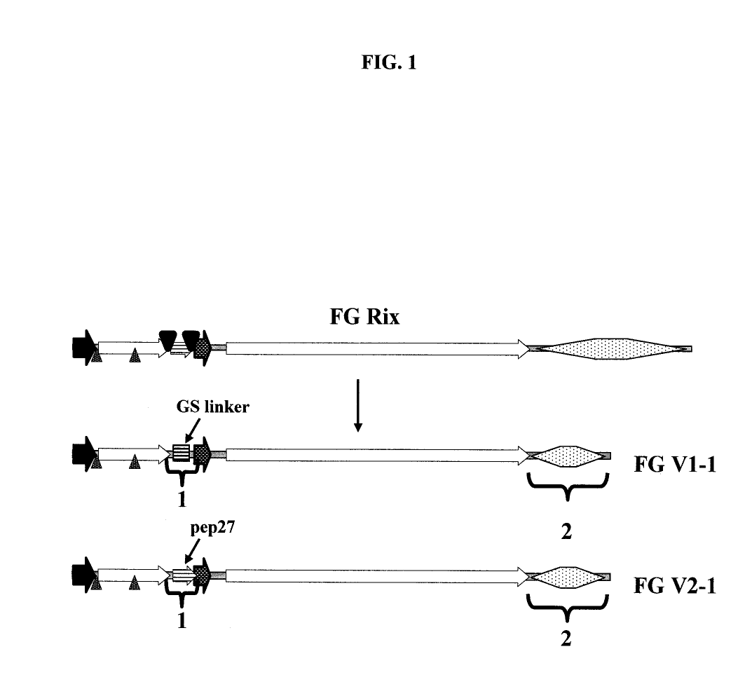

FIG. 2 is a sequence alignment providing a comparison of FG-Rix and two exemplary novel improved FG chimeras designated FG V1-1 and FG V2-1.

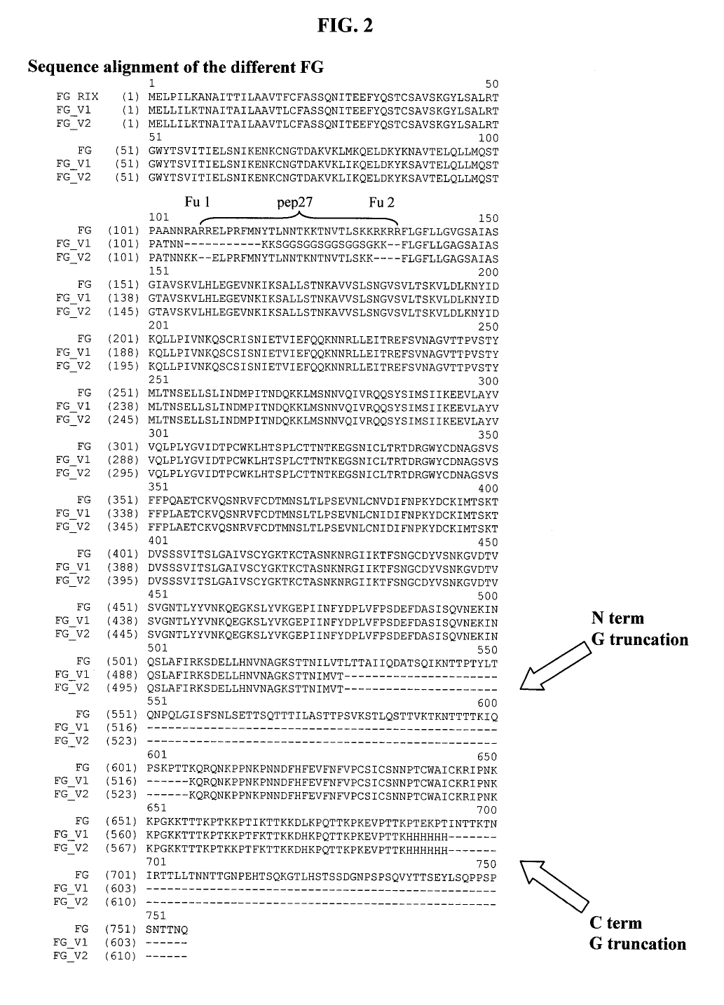

FIGS. 3A and B are bar graphs illustrating neutralization inhibition of human sera by FG V1-1 and FG V2-1.

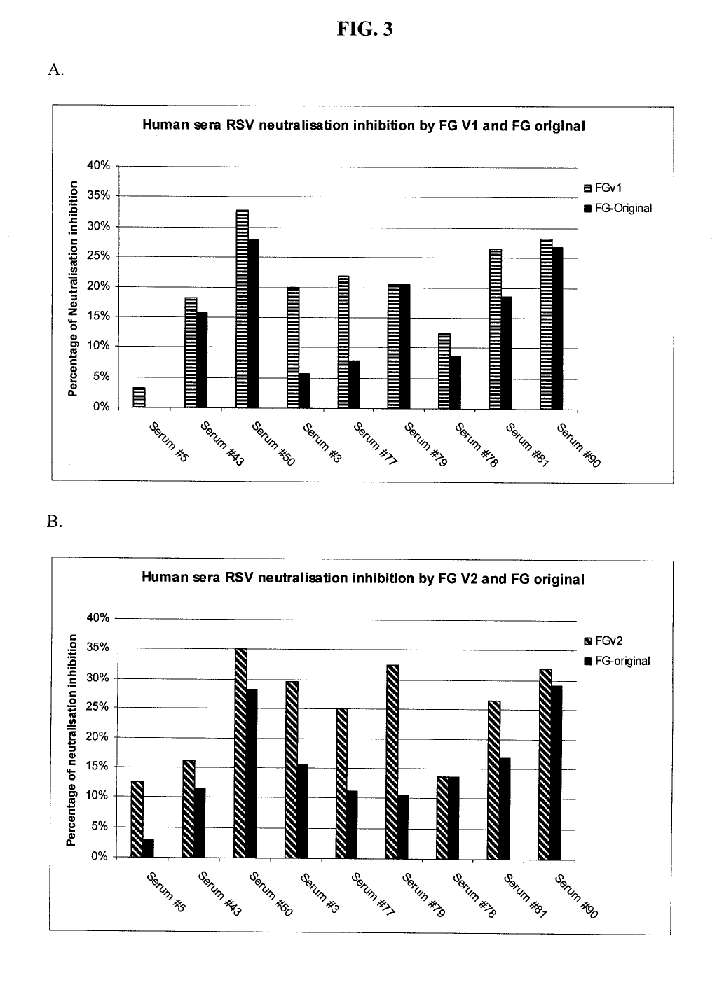

### DETAILED DESCRIPTION

### Introduction

Development of vaccines that protect against the symptoms and sequelae caused by RSV infection has been complicated by the fact that host immune responses appear to play a role in the pathogenesis of the disease. Early studies in the 1960s showed that children vaccinated with a formalin-inactivated RSV vaccine suffered from more severe disease on subsequent exposure to the virus as compared to unvaccinated control subjects. These early trials resulted in the hospitalization of 80% of vaccinees and two deaths. The enhanced severity of disease has been reproduced in animal models and is thought to result from inadequate levels of serum-neutralizing antibodies, lack of local immunity, and excessive induction of a type 2 helper T-cell-like (Th2) immune response with pulmonary eosinophilia and increased production of IL-4 and IL-5 cytokines. In contrast, a successful vaccine that protects against RSV infection induces a Th1-type immune response, characterized by production of IL-2 and γ-interferon (IFN-γ).

Various approaches, including killed or inactivated virus, attenuated live virus and purified subunit approaches, have been attempted in efforts to produce a safe and effective RSV vaccine that produces durable and protective immune responses in healthy and at risk populations. However, none of the candidates evaluated to date have resulted in the marketing of a vaccine for the purpose of preventing RSV infection and/or reducing or preventing RSV disease. One approach has involved the production of recombinant chimeric antigens that include components of both the RSV Fusion (F) and Attachment (G) glycoproteins. Exemplary chimeric RSV antigens are disclosed in U.S. Pat. No. 5,194,595. These chimeric constructs included the entire extracellular domains of the RSV F and G proteins (i.e., amino acid residues 1-526 of RSV F, and 69-298 of RSV G). Although this chimeric antigen elicited an immune response in animal models (e.g., mice, cotton rats), it was unable to proceed to market due to production and stability difficulties.

The present disclosure concerns novel chimeric FG polypeptides with excellent immunogenicity and superior process characteristics. These novel chimeric RSV antigens overcome several significant drawbacks encountered in previous attempts to produce safe and effective chimeric RSV antigens that are suitable for administration as prophylactic and therapeutic vaccines.

In one aspect, the disclosure relates to a respiratory syncytial virus (RSV) antigen that includes a chimeric polypeptide comprising in an N terminal to C terminal direction (i) a first amino acid sequence that includes an F2 domain uncleavably joined to an F1 domain of a F protein polypeptide; and (ii) a second amino acid sequence that includes a portion of a G protein polypeptide containing an immunologically dominant epitope. Typically, the F2 domain and the F1 domain of the RSV F protein polypeptide are uncleavably joined via an amino acid linker. The F2 and F1 domains can be joined in an uncleavable manner by eliminating the furin cleavage recognition sequence and/or sites that render the F2 and F1 domains separable and result in the release of the pep27 peptide during maturation and assembly of a native F protein. For example, the chimeric RSV polypeptide can in clued at least one amino acid deletion or substitution that removes a furin cleavage site, thereby rendering the chimeric polypeptide uncleavable. For example, one or more amino acids (e.g., at positions 106 and 133) can be deleted or substituted to produce an uncleavable F protein. In certain illustrative embodiments two amino acids (including at least one arginine, e.g., arginine and alanine at positions 106 and 107, and arginine and lysine at positions 133 and 134) can be deleted or substituted.

Optionally, to facilitate expression and recovery, the chimeric RSV polypeptide includes a signal peptide at the N-terminus. A signal peptide can be selected from among numerous signal peptides known in the art, and is typically chosen to facilitate (enhance or maximize) production and processing in a system selected for recombinant expression of the chimeric polypeptide. Signal peptides are generally in the range of 18-25 amino acids in length. In certain embodiments, the signal peptide is from an RSV F protein, e.g., amino acid residues 1-23 of SEQ ID NO:2. The F2 domain can include amino acid residues 24-105 of a native F protein polypeptide. It will be apparent that the precise amino acid limits between the signal peptide and the F2 domain can be varied by one or more amino acids (indeed, in the case of a signal peptide selected from an RSV F protein, such a limit is arbitrary). In exemplary embodiments, the F1 domain includes essentially all of the extracellular portion of the F1 domain, e.g., from residue 137 to residue 528 of a native F protein polypeptide. As indicated above, the F2 and F1 domains can be uncleavable joined by an amino acid linker sequence. Numerous amino acid linkers are known to those of skill in the art an suitable in the context of the chimeric FG polypeptides disclosed herein. In exemplary embodiments, the linker is selected from the following sequences: SEQ ID NO:5, SEQ ID NO:6, SEQ ID NO:7 and SEQ ID NO:8.

The F polypeptide sequence is joined in frame to a portion of the RSV G protein polypeptide. A portion of the G protein is selected to improve production characteristics (for example, as compared to a full-length G protein polypeptide). The portion of the G protein is selected to retain immunologically dominant epitopes, in particular, an immunologically dominant epitope between amino acid residues 183 and 203. For example, the portion of the RSV G protein includes amino acids 152-229. In certain exemplary embodiments, the portion of the RSV G protein includes amino acids residues 149-229 of the G protein.

The sequences of the F and G components can be selected from naturally occurring F and G protein sequences, and can be selected to correspond in sequence to a single strain or to more than one strain. For example, the F and G portions can be from the same strain, or the F and G portions can each be from a different strain, or the F portion or the G portion or both can be a hybrid corresponding to amino acids from more than one strain. Optionally, the chimeric RSV polypeptide can include one or more than one amino acid substitutions relative to a naturally occurring RSV polypeptide. For example, an amino acid substitution can be introduced to into the G protein portion, such as an amino acid substitution that is correlated with reduction or prevention of vaccine enhanced viral disease in a model system, e.g., the chimeric polypeptide can include a substitution of asparagine by alanine at residue 191 (N191A) of the G protein.

Optionally, the chimeric RSV polypeptides as disclosed herein can include a polyhistidine tag, or another such sequence designed to facilitate or enhance recovery and/or purification of a recombinantly expressed protein.

In certain exemplary embodiments, the chimeric RSV polypeptide has an amino acid sequence selected from SEQ ID NOs:11 or 13, or a subsequence thereof, (e.g., a subsequence lacking the signal sequence of amino acids 1-23, or having a substitution of a different signal sequence and/or lacking the C terminal histidine tag). Favorably, the chimeric RSV polypeptide includes at least one immunodominant epitiope of both the RSV F protein and the RSV G protein.

Upon expression (e.g., and purification or isolation), the chimeric RSV polypeptide assembles into a multimer that possesses a conformation that immunologically resembles the native F protein. For example, the chimeric RSV polypeptide can favorably assemble into a trimer.

Also encompassed by this disclosure are immunogenic compositions that include any of the chimeric RSV polypeptides described above, formulated with a carrier or excipient. Typically, the carrier or excipient is a pharmaceutically acceptable carrier or excipient, such as a buffer. Optionally, the carrier or excipient can include additional components that enhance stability, solubility or both stability and solubility of the chimeric RSV polypeptide. Optionally, the immunogenic composition further comprises an adjuvant suitable for administration into the population of subjects for which the composition is intended to be administered e.g., to prevent, reduce or ameliorate RSV induced disease or symptoms. Accordingly, the adjuvant can be selected for administration to a neonate, an infant or an adult, such as an adult of at least 65 years of age. Favorably, the adjuvant is a Th1-biasing adjuvant. In certain embodiments, the adjuvant is a TLR-4 ligand, such as 3D-MPL, or any other synthetic derivative of lipid A. Optionally, the immunogenic composition can also include a particulate carrier, such as alum. In certain embodiments, the adjuvant can include a liposome or an emulsion, e.g., an oil-in-water emulsion.

The immunogenic composition is favorably formulated for use as a medicament in humans, e.g., for the prevention or reduction of infection with RSV following administration to a human subject, or for the prevention or reduction of a pathological response caused by infection with RSV following administration to a human subject. Optionally, the immunogenic composition also includes at least one additional antigen of a pathogenic organism other than RSV. For example, the pathogenic organism can be a virus other than RSV, such as Parainfluenza virus (Hy), influenza virus, hepatitis B virus, and/or poliovirus. Alternative, the pathogenic organism can be a bacteria, such as diphtheria, tetanus, pertussis, Hemophilus influenza, and/or Pneumococcus.

Another aspect of this disclosure concerns recombinant nucleic acids encode any of the chimeric polypeptides provided herein. In some embodiments, the nucleic acids include polynucleotide sequence that have been codon optimized for expression in a selected host cell (e.g., codon optimized for expression in an a mammalian cell, a yeast cell, a plant cell, etc.). In some cases, the nucleic acids are contained within vectors, such as a prokaryotic or eukaryotic expression vector. Cells into which such nucleic acids or vectors are introduced (that is, host cells) are also a feature of this disclosure. The host cells can be bacterial cells, but more commonly will be eukaryotic cells, such as yeast cells (e.g., picchia), plant cells, insect cells, or mammalian cells (e.g., CHO cells).

The chimeric polypeptides and nucleic acids are useful in the preparation of medicaments for treating (e.g., prophylactically) an RSV infection. Accordingly, this disclosure also provides methods for eliciting an immune response against RSV by administering an immunologically effective amount of a composition containing any of the the chimeric RSV polypeptides disclosed herein. Favorably, when administered to a human subject (such as as a neonate, infant or child or to an elderly subject) the composition elicits an immune response specific for RSV without enhancing viral disease following contact with RSV. Favorably, the composition elicits a protective immune response that reduces or prevents infection with a RSV and/or reduces or prevents a pathological response following infection with a RSV. Typically, the immune response elicits an immune response characterized by the production of Th1 type cytokines, e.g., a Th1-type immune response.

### Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of terms are provided. Additional terms and explanations can be provided in the context of this disclosure.

Respiratory syncytial virus (RSV) is a pathogenic virus of the family Paramyxoviridae, subfamily Pneumovirinae, genus Pneumovirus. The genome of RSV is a 15,222 nucleotide-long, single-stranded, negative-sense RNA molecule, which encodes 11 proteins. Tight association of the RNA genome with the viral N protein forms a nucleocapsid wrapped inside the viral envelope. Two groups of human RSV strains have been described, the A and B groups, based on differences in the antigenicity of the G glycoprotein. Numerous strains of RSV have been isolated to date. Exemplary strains are indicated by GenBank and/or EMBL Accession number in FIGS. 4 and 5. Additional strains of RSV are likely to be isolated, and are encompassed within the genus of RSV. Similarly, the genus of RSV encompasses variants arising from naturally occurring (e.g., previously or subsequently identified strains) by genetic drift, or artificial synthesis and/or recombination.

The term “F protein” or “Fusion protein” or “F protein polypeptide” or Fusion protein polypeptide” refers to a polypeptide or protein having all or part of an amino acid sequence of an RSV Fusion protein polypeptide. The term “G protein” or “G protein polypeptide” refers to a polypeptide or protein having all or part of an amino acid sequence of an RSV Attachment protein polypeptide. Numerous RSV Fusion and Attachment proteins have been described and are known to those of skill in the art.

To facilitate understanding of this disclosure, when referring to amino acid residue positions of RSV F and/or G proteins, all amino acid residue positions are given with reference to (that is, the amino acid residue position corresponds to) the amino acid position of the exemplary F protein of SEQ ID NO:2, and to the amino acid positions of the exemplary G protein of SEQ ID NO: 4. However, comparable amino acids can be used from any RSV A or B strain. Comparable amino acid positions of any other RSV A or B strain can be determined easily by those of ordinary skill in the art by aligning the amino acid sequences of the selected RSV strain with that of SEQ ID NO:2 using readily available and well-known alignment algorithms (such as BLAST, e.g., using default parameters) Exemplary F and G protein sequences from numerous strains are provided in WO2008114149, any of which can be employed in the context of the chimeric FG proteins disclosed herein. WO2008114149 is incorporated herein by reference for the purpose of disclosing the sequences of RSV F and G proteins suitable for use in chimeric G proteins.

A “chimeric FG polypeptide” or an “FG antigen” or “FG polypeptide antigen” is a chimeric polypeptide that incorporates polypeptide components, typically including antigenic determinants or epitopes of both an RSV F protein and an RSV G protein. In the context of this disclosure, the chimeric FG polypeptides include in an N-terminal to C-terminal orientation: a first amino acid sequence that includes an F2 domain uncleavably joined to an F1 domain of and a second amino acid sequence that includes a portion of the RSV G protein containing an immunologically dominant epitope. The term subunit and domain are used interchangeably in reference to structural domains of the F protein and/or F0 polypeptide. The term chimeric in this context includes polypeptides in which the F and G protein components are both from the same serotype or strain, as well as polypeptides in which the individual F and G protein components are from different serotypes or strains.

A “variant” when referring to a nucleic acid or a protein (e.g., an RSV F or G protein or protein domain, or an FG chimeric polypeptide) is a nucleic acid or a polypeptide that differs from a reference nucleic acid or protein. Usually, the difference(s) between the variant and the reference nucleic acid or protein constitute a proportionally small number of differences as compared to the reference. Such differences can be amino acid additions, deletions or substitutions. Thus, a variant typically differs by no more than about 1%, or 2%, or 5%, or 10%, or 15%, or 20% of the nucleotide or amino acid residues. Thus, a variant in the context of an RSV F or G protein, or a chimeric FG polypeptide, typically shares at least 80%, or 85%, more commonly, at least about 90% or more, such as 95%, or even 98% or 99% sequence identity with a reference protein, e.g. the reference sequences illustrated in SEQ ID NO:2 and 4, or any of the exemplary FG polypeptides disclosed herein. Additional variants included as a feature of this disclosure are chimeric FG polypeptides that incorporate an F2 (e.g., comprising all or part of amino acids 24-105, numerically designated by alignment with SEQ ID NO:2) and/or F1 component (e.g., comprising all or part of amino acids 137-528, numerically designated by alignment with SEQ ID NO:2) from any of the exemplary sequences, e.g., provided in WO2008114149 (either the same or different strain) and a G protein component (e.g., all or part of amino acids 149-229, numerically designated by alignment to SEQ ID NO:4) selected from any of the exemplary sequences, e.g., provided in WO2008114149. Variants can arise through genetic drift, or can be produced artificially using site directed or random mutagenesis, or by recombination of two or more preexisting variants. For example, a variant FG polypeptide can include 1, or 2, or 5 or 10, or 15, or 50 amino acid differences as compared to the exemplary FG chimeras of SEQ ID NOs: 11 and 13, or up to about 100 nucleotide differences as compared to the exemplary FG chimeric nucleic acids, e.g., of SEQ ID NOs:10 and 12.

A “domain” of a polypeptide or protein is a structurally defined element within the polypeptide or protein. In the context of this disclosure, a “furin cleavage domain” is a domain defined by cleavage of a precursor polypeptide by a furin protease. For example, the F protein is synthesized as a single polypeptide, designated F0. The F0 polypeptide is subsequently cleaved at two consensus furin recognition motifs by a furin protease to produce two structurally independent polypeptide units designated F2 and F1. F2 extends from amino acid 24 (following the signal peptide) to the first (in an N- to C-terminal direction) furin cleavage recognition site. F1 extends from the second furin cleavage site to the C-terminal end of the F0 polypeptide. In the context of this disclosure, the term F1 is also used to refer to a portion of the F0 polypeptide that includes the extracellular portion of the F1 domain (e.g., amino acids 137-528).

The terms “native” and “naturally occurring” refer to an element, such as a protein, polypeptide or nucleic acid, that is present in the same state as it is in nature. That is, the element has not been modified artificially. It will be understood, that in the context of this disclosure, there are numerous native/naturally occurring variants of RSV proteins or polypeptides, e.g., obtained from different naturally occurring strains or isolates of RSV.

The term “polypeptide” refers to a polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “fragment,” in reference to a polypeptide, refers to a portion (that is, a subsequence) of a polypeptide. The term “immunogenic fragment” refers to all fragments of a polypeptide that retain at least one predominant immunogenic epitope of the full-length reference protein or polypeptide. Orientation within a polypeptide is generally recited in an N-terminal to C-terminal direction, defined by the orientation of the amino and carboxy moieties of individual amino acids. Polypeptides are translated from the N or amino-terminus towards the C or carboxy-terminus.

A “signal peptide” is a short amino acid sequence (e.g., approximately 18-25 amino acids in length) that direct newly synthesized secretory or membrane proteins to and through membranes, e.g., of the endoplasmic reticulum. Signal peptides are frequently but not universally located at the N-terminus of a polypeptide, and are frequently cleaved off by signal peptidases after the protein has crossed the membrane. Signal sequences typically contain three common structural features: an N-terminal polar basic region (n-region), a hydrophobic core, and a hydrophilic c-region).

The terms “polynucleotide” and “nucleic acid sequence” refer to a polymeric form of nucleotides at least 10 bases in length. Nucleotides can be ribonucleotides, deoxyribonucleotides, or modified forms of either nucleotide. The term includes single and double forms of DNA. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. In one embodiment, a polynucleotide encodes a polypeptide. The 5′ and 3′ direction of a nucleic acid is defined by reference to the connectivity of individual nucleotide units, and designated in accordance with the carbon positions of the deoxyribose(or ribose) sugar ring. The informational (coding) content of a polynucleotide sequence is read in a 5′ to 3′ direction.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the host cell chromosome.

The term “purification” (e.g., with respect to a pathogen or a composition containing a pathogen) refers to the process of removing components from a composition, the presence of which is not desired. Purification is a relative term, and does not require that all traces of the undesirable component be removed from the composition. In the context of vaccine production, purification includes such processes as centrifugation, dialization, ion-exchange chromatography, and size-exclusion chromatography, affinity-purification or precipitation. Thus, the term “purified” does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid or protein can be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid or protein content of the preparation.

An “isolated” biological component (such as a nucleic acid molecule, protein or organelle) has been substantially separated or purified away from other biological components in the cell of the organism in which the component naturally occurs, such as, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “immunologically dominant” epitopes are those epitopes to which a functionally significant host immune response, e.g., an antibody response or a T-cell response, is made. Thus, with respect to a protective immune response against a pathogen, the immunologically dominant epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

An “adjuvant” is an agent that enhances the production of an immune response in a non-specific manner. Common adjuvants include suspensions of minerals (alum, aluminum hydroxide, aluminum phosphate) onto which antigen is adsorbed; emulsions, including water-in-oil, and oil-in-water (and variants therof, including double emulsions and reversible emulsions), liposaccharides, lipopolysaccharides, immunostimulatory nucleic acids (such as CpG oligonucleotides), liposomes, Toll Receptor agonists (particularly, TLR2, TLR4, TLR7/8 and TLR9 agonists), and various combinations of such components.

An “immunogenic composition” is a composition of matter suitable for administration to a human or animal subject that is capable of eliciting a specific immune response, e.g., against a pathogen, such as RSV. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., RSV) following exposure of the subject to the pathogen. In the context of this disclosure, the term immunogenic composition will be understood to encompass compositions that are intended for administration to a subject or population of subjects for the purpose of eliciting a protective or palliative immune response against RSV (that is, vaccine compositions or vaccines).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

A “Th1” type immune response is characterized CD4+ T helper cells that produce IL-2 and IFN-γ. In contrast, a “Th2” type immune response is characterized by CD4+ helper cells that produce IL-4, IL-5, and IL-13.

A “immunologically effective amount” is a quantity of a composition (typically, an immunogenic composition) used to elicit an immune response in a subject. Commonly, the desired result is the production of an antigen (e.g., pathogen)-specific immune response that is capable of or contributes to protecting the subject against the pathogen. However, to obtain a protective immune response against a pathogen can require multiple administrations of the immunogenic composition. Thus, in the context of this disclosure, the term immunologically effective amount encompasses a fractional dose that contributes in combination with previous or subsequent administrations to attaining a protective immune response.

The adjective “pharmaceutically acceptable” indicates that the subject is suitable for administration to a subject (e.g., a human or animal subject). Remington's Pharmaceutical Sciences, by E. W. Martin, Mack Publishing Co., Easton, Pa., 15th Edition (1975), describes compositions and formulations (including diluents) suitable for pharmaceutical delivery of therapeutic and/or prophylactic compositions, including immunogenic compositions.

The term “modulate” in reference to a response, such as an immune response, means to alter or vary the onset, magnitude, duration or characteristics of the response. An agent that modulates an immune response alters at least one of the onset, magnitude, duration or characteristics of an immune response following its administration, or that alters at least one of the onset, magnitude, duration or characteristic as compared to a reference agent.

The term “reduces” is a relative term, such that an agent reduces a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, an immunogenic composition that reduces or prevents an infection or a response, such as a pathological response, e.g., vaccine enhanced viral disease, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

A “subject” is a living multi-cellular vertebrate organism. In the context of this disclosure, the subject can be an experimental subject, such as a non-human animal, e.g., a mouse, a cotton rat, or a non-human primate. Alternatively, the subject can be a human subject.

### FG Chimeric RSV Antigens

The viral envelope of RSV includes virally encoded F, G and SH glycoproteins. The F and G glycoproteins are the only two components of the RSV virion that are known to induce RSV-specific neutralizing antibodies. The chimeric FG polypeptides disclosed herein are designed to incorporate structural features of the native F protein while simultaneously exhibiting important immunodominant epitopes of the RSV G protein.

The native F protein of RSV is translated as a single polypeptide precursor, designated F0. F0 folds and is subject to proteolysis and other post-translational modifications. First, a signal peptide (Sp) targets the translation of the nascent polypeptide to the endoplasmic reticulum (ER) and is later cleaved by a signal peptidase. The nascent polypeptide is then N-glycosylated in the RE at 3 sites, at amino acid positions 27, 70 and 500 of the exemplary F polypeptide sequence of SEQ ID NO:2. F2 and F1 are generated by furin-cleavage and folded together as a trimer of heterodimer (3 times F2-F1). Furin is a calcium-dependent serine endoprotease that can efficiently cleave precursor proteins at paired basic amino acid processing sites. Typically, such processing sites include a basic amino acid target sequence (canonically, Arg-X-(Arg/Lys)-Arg′). The RSV F protein includes two furin recognition sites at positions 106-109 and 133-136. Proteolytic cleavage of the naturally occurring mature F0 polypeptide by a furin protease at the two conserved furin consensus sequences, RAR/KR¹⁰⁹ (FCS-2) and KKRKRR¹³⁶ (FCS-1), results in the generation of three proteolytic fragments. The large membrane-anchored subunit F1 with a hydrophobic fusion peptide at its N terminus (corresponding to amino acids 137-574) is linked to the small subunit F2 (corresponding to amino acids 24-105) via a disulfide bridge, and a small peptide composed of 27 amino acids (pep27) originally located between the two cleavage sites is released. It will be recognized by those of skill in the art that the abbreviations F0, F1 and F2 are commonly designated F₀, F₁ and F₂ in the scientific literature. A description of furin processing of the RSV F protein, along with definitions of the art-accepted terminology is found in Zimmer et al. “Proteolytic activation of Respiratory Syncytial Virus fusion protein.” J. Biol. Chem. 276:31642-31650, 2001, and Zimmer et al., “Cleavage at the furin consensus sequence RAR/KR109 and presence of the intervening peptide of the Respiratory Syncytial Virus fusion protein are dispensable for virus replication in cell culture.” J. Virol. 76:9218-9224, 2002. The protein is anchored to the membrane using its transmembrane helix shown by the white lozenge (TM) in the C-terminal region. In addition, the RSV F protein features 15 Cysteines residues, 4 characterized neutralizing epitopes, 2 coiled-coil regions and a lipidation motif.

The native G protein is 298 amino acid protein that is anchored to the virion membrane by its transmembrane hydrophobic region (amino acids 41-63). Amino acids 65-298 includes the portion of the G protein that is exposed at the surface of RSV. Highly O-glycosylated mucin-like regions are present at each extremity. Five N-glycosylation motifs are also present in these two regions. The non-glycosylated central includes several important structural motifs, including: 1) a cysteine noose (aa173-190), which is the only portion of the G for which structural data are available; 2) an immunodominant MHC class II epitope at aal 83-203; and 3) chemokine fractalkine receptor (C3XCR) and glycosaminoglycan (GAG) binding motifs, which are implicated in the process of viral attachment on the host cell surface.

This disclosure concerns chimeric RSV polypeptides that include in an N-terminal to C-terminal orientation: (i) a first amino acid sequence that includes an F2 domain joined to an F1 domain of the RSV F protein and (ii) a second amino acid sequence that includes a portion of an RSV G protein. To facilitate folding and assembly during production, the native F protein sequence is modified to eliminate internal furin recognition sites and prevent furin cleavage. The furin cleavage sites can be destroyed by the addition, deletion or substitution of one or more amino acids in the region of amino acid residues 106-109 and/or 133-136. For example, the furin recognition sites can be eliminated by deleting one or two amino acids (for example arginine and alanine at positions 106 and 107, and arginine and lysing at positions 133 and 134), that destroy the furin cleavage sites. Thus, upon expression and assembly, the F2 and F1 portions of the chimeric polypeptide remain in a single uncleavable polypeptide unit.

In selecting F2 and F1 domains of the F protein, one of skill in the art will recognize that it is not strictly necessary to include the entire F2 and/or F1 domain. Typically, conformational considerations are of importance when selecting a subsequence (or fragment) of the F2 domain. Thus, the F2 domain typically includes a portion of the F2 domain that facilitates assembly and stability of the chimeric polypeptide. In certain exemplary variants, the F2 domain includes amino acids 24-105. Optionally, the F2 domain can include a signal peptide of the native F0 polypeptide (e.g., amino acids 1-23).

Typically, at least a subsequence (or fragment) of the F1 domain is selected and designed to maintain a stable conformation that includes immunodominant epitopes of the F protein. For example, it is generally desirable to select a subsequence of the F1 polypeptide domain that includes epitopes recognized by neutralizing antibodies in the regions of amino acids 262-275 (palivizumab neutralization) and 423-436 (Centocor's ch101F MAb). Additionally, desirable to include T cell epitopes, e.g., in the region of animo acids 328-355. Most commonly, as a single contiguous portion of the F1 subunit (e.g., spanning amino acids 262-436) but epitopes could be retained in a synthetic sequence that includes these immunodominant epitopes as discontinuous elements assembled in a stable conformation. Thus, an F1 domain polypeptide comprises at least about amino acids 262-436 of an RSV F protein polypeptide. In one non-limiting example provided herein, the F1 domain comprises amino acids 137 to 528 of a native F protein polypeptide (although somewhat smaller fragments could be employed, for example, a fragment beginning at amino acid residue 151 or amino acid 161, or terminating at position 524). One of skill in the art will recognize that additional shorter subsequences can be used at the discretion of the practitioner.

To facilitate folding and assembly, and maximize retention of conformational epitopes, an amino acid linker is introduced between the two F protein domains. Numerous linkers of varying length and structural attributes are known to those of skill in the art. In the context of the chimeric RSV polypeptides disclosed herein, any of a number of such linkers can be employed. For example, simple glycine-rich repeated sequences are favorably employed as linkers, as illustrated in the embodiments designated V1-1 and V1-2. In FG V1-1, a simple glycine and serine repeat sequence is employed as a linker. The variant of FG V1-2 includes a glycine/serine linker that is adapted to include a glycosylation site. Specific exemplary glycine/serine linker sequences are provided in SEQ ID NOs:5 and 6, respectively. Alternatively, linkers with more complex structural attributes can be employed. In certain embodiments, a linker is selected from the native F protein. For example, in certain favorable embodiments, the linker corresponds in sequence to all or part of the pep27 sequence, as illustrated by embodiments designated V2-1 and V2-2. Where such a linker is employed, it can be varied in length, e.g., to modify structural or functional characteristics, such as glycosylation. Two exemplary versions of a pep27-based linker are provided in SEQ ID NOs:7 and 8.

The G protein polypeptide component is selected to include a portion (or subsequence or fragment) of the G protein that retains the immunologically dominant or immunodominant T cell epitope(s), e.g., in the region of amino acids 183-197. Exemplary variants include, for example subsequences or fragments of the G protein that include from amino acids 152, 151, 150, 149, 148, etc. to amino acids 226, 227, 228, 229, 230, etc. Optionally, a larger fragment (such as a fragment including amino acid residues 128-229, or 130-230) of a native G protein can be substituted. One of skill in the art will readily appreciate that longer or shorter portions of the G protein can also be used, so long as the portion selected does not conformationally destabilize or disrupt expression, folding or processing of the chimeric FG polypeptide. Optionally, the G protein domain includes an amino acid substitution at position 191, which has previously been correlated with reducing and/or preventing enhanced disease characterized by eosinophilia associated with formalin inactivated RSV vaccines. A thorough description of the attributes of naturally occurring and substituted (N191A) G proteins can be found, e.g., in US Patent Publication No. 2005/0042230, which is incorporated herein by reference for all purposes.

If so desired, additional T cell epitopes can be identified using anchor motifs or other methods, such as neural net or polynomial determinations, known in the art, see, e.g., RANKPEP (available on the world wide web at: mif.dfci.harvard.edu/Tools/rankpep.html); ProPredI (available on the world wide web at: imtech.res.in/raghava/propredI/index.html); Bimas (available on the world wide web at: www-bimas.dcrt.nih.gov/molbi/hla_bind/index.html); and SYFPEITH (available on the world wide web at: syfpeithi.bmi-heidelberg.com/scripts/MHCServer.dll/home.htm). For example, algorithms are used to determine the “binding threshold” of peptides, and to select those with scores that give them a high probability of MHC or antibody binding at a certain affinity. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. Anchor residues are conserved residues that provide a contact point with the MHC molecule. T cell epitopes identified by such predictive methods can be confirmed by measuring their binding to a specific MHC protein and by their ability to stimulate T cells when presented in the context of the MHC protein.

Although exemplary embodiments are set forth in SEQ ID NOs: 11 and 13, numerous other embodiments can be produced without undue experimentation by those of ordinary skill in the art. It will be evident to those of skill in the art that any RSV F and/or G protein sequences can be employed in the construction of recombinant chimeric RSV FG polypeptides. The sequence of the F protein, which is responsible for fusion of the virus envelope with the target cell membrane, is highly conserved among RSV isolates. In contrast, that of the G protein, which is responsible for virus attachment, is relatively variable. An alignment of RSV F and G protein sequences, illustrating identity and variation between the different proteins, are provided WO2008114149. Conserved and variable regions are readily apparent from these alignments.

For example, in one embodiment, the F2 domain (e.g., corresponding to amino acids 24-105 of the reference F protein sequence) are uncleavably joined to an F1 domain (e.g., corresponding to amino acids 137-528 of the reference F protein sequence) by a linker selected from any of SEQ ID NOs: 5, 6, 7, or 8, and joined in frame to a G protein domain that includes the immunodominant epitope provided by amino acids 183-203 of the G protein (e.g., from approximately the amino acid corresponding to position 149 to approximately the amino acid corresponding to position 229 of the reference F protein sequence, for example, from position 148, 149, 150, 151 or 152 to position 226, 227, 228, 229 or 230). The F2 and F1 domains can be selected from the same F protein polypeptide, such as an F protein polypeptide selected from a naturally occurring F protein such as that of SEQ ID NO:2, or any of the other exemplary F protein polypeptides (for example, those disclosed in WO2008114149). Alternatively, the F2 and F1 domains can be selected from different naturally occurring F protein polypeptides. Alternatively, one or both of the F2 and F1 domains can be modified as indicated in more detail in the discussion herein regarding variants. Similarly, the G protein domain can be selected from SEQ ID NO:4 or from any of the variants disclosed in WO2008114149.

Other exemplary embodiments are variants that have a deletion of one or more amino acids. Where shorter fragments are desired, a portion is nonetheless selected that retains structurally and immunologically important features of the components of the chimeric polypeptide, as described herein. Alternatively, variants can include additional amino acids. For example, the variants can include additional amino acids, which facilitate purification, (e.g., polyhistidine tags).

Additionally, or alternatively, modifications can be made to any of the disclosed chimeric FG polypeptides to enhance expression and stability of the chimeric polypeptides when produced in a selected expression system. For example, eukaryotic constructs are typically designed to include a signal peptide corresponding to the expression system, for example, a mammalian or viral signal peptide, such as the RSV F0 native signal sequence is favorably selected when expressing the chimeric polypeptide in mammalian cells. Alternatively, a signal peptide (such as a baculovirus signal peptide, or the melittin signal peptide, can be substituted for expression, in insect cells. Suitable plant signal peptides are known in the art, if a plant expression system is preferred. Exemplary signal peptides suitable for use in the context of the chimeric FG polypeptides disclosed herein include signal peptides of: tissue plasminogen activator (tPA), Herpes Simplex Virus (HSV) gD protein, human endostatin, HIV gp120, CD33, cytomegalovirus gB protein, human Her2Neu, Epstein Barr Virus (EBV) gp350, and the SS of Tan et al., Protein Eng. 15:337-45.

In certain embodiments, the chimeric FG polypeptides are modified to alter the glycosylation pattern or status (e.g., by increasing or decreasing the proportion of molecules glycosylated at one or more of the glycosylation sites present in a native F protein polypeptide. For example, the native F protein polypeptide of SEQ ID NO:2 is predicted to be glycosylated at amino acid positions 27, 70 and 500. In an embodiment, a modification is introduced in the vicinity of the glycosylation site at amino acid position 500. For example, the glycosylation site can be removed by substituting an amino acid, such as glutamine (Q) in place of the asparagine at the position corresponding to position 500 of the reference F protein sequence (SEQ ID NO:2). Favorably, a modification that increases glycosylation efficiency at this glycosylation site is introduced. Examples of suitable modifications include at positions 500-502, the following amino acid sequences: NGS; NKS; NGT; NKT. Modifications of this glycosylation site that result in increased glycosylation can also result in substantially increased protein production.

Nucleic Acids that Encode Chimeric FG Polypeptide Antigens

Another aspect of this disclosure concerns recombinant nucleic acids that encode the chimeric FG polypeptides described above. The recombinant nucleic acids include in a 5′ to 3′ direction, (1) a polynucleotide sequence that encodes at least a portion or fragment of an RSV F protein polypeptide furin cleavage domain 2 (F2 domain); (2) a polynucleotide sequence that encodes an amino acid linker; (3) a polynucleotide sequence that encodes at least a portion or fragment of an RSV F protein polypeptide furin cleavage domain 1 (F1 domain); and (4) a polynucleotide sequence that encodes at least a portion or fragment of an RSV G protein polypeptide. The component polynucleotide sequences are joined such that the encoded polypeptide segments are produced in a single contiguous chimeric polypeptide as disclosed above.

In certain embodiment, the recombinant nucleic acid encode a chimeric FG polypeptide in which the F2 domain (e.g., corresponding to amino acids 24-105 of the reference F protein sequence) is uncleavably joined to an F1 domain (e.g., corresponding to amino acids 137-528 of the reference F protein sequence) by a linker selected from any of SEQ ID NOs: 5, 6, 7, or 8, and joined in frame to a G protein domain that includes the immunodominant epitope provided by amino acids 183-203 of the G protein (e.g., from approximately the amino acid corresponding to position 149 to approximately the amino acid corresponding to position 229 of the reference F protein sequence, for example, from position 148, 149, 150, 151 or 152 to position 226, 227, 228, 229 or 230). The polynucleotides that encode the F2 and F1 domains can be selected from the same F protein polypeptide, such as an F protein polypeptide selected from a naturally occurring F protein such as that of SEQ ID NO:2 (e.g., SEQ ID NO:1), or that encode any of the other exemplary F protein polypeptides (for example, those disclosed in WO2008114149). Alternatively, the F2 and F1 domains can be selected to encode different naturally occurring F protein polypeptides. Alternatively, the polynucleotides that encode one or both of the F2 and F1 domains can include one or more mutations (e.g., nucleotide addition, deletion or substitution) to modify the polypeptide as indicated (for example, to modify a glycosylation site at position 27, 70 and/or 500) in more detail in the discussion herein regarding variants. Similarly, the polynucleotide that encodes the G protein domain can be selected from SEQ ID NO:4 or from any of the variants disclosed in WO2008114149.

In certain embodiments, the recombinant nucleic acids are codon optimized for expression in a selected prokaryotic or eukaryotic host cell, such as a mammalian, plant or insect cell. To facilitate replication and expression, the nucleic acids can be incorporated into a vector, such as a prokaryotic or a eukaryotic expression vector. Although the nucleic acids disclosed herein can be included in any one of a variety of vectors (inelding, for example, bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others), most commonly the vector will be an expression vector suitable for generating polypeptide expression products. In an expression vector, the nucleic acid encoding the FG chimera is typically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: the immediate early promoter of CMV, LTR or SV40 promoter, polyhedron promoter of baculovirus, E. coli lac or trp promoter, phage T7 and lambda P_{L} promoter, and other promoters known to control expression of genes in prokaryotic or eukaryotic cells or their viruses. The expression vector typically also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The expression vector can also include additional expression elements, for example, to improve the efficiency of translation. These signals can include, e.g., an ATG initiation codon and adjacent sequences. In some cases, for example, a translation initiation codon and associated sequence elements are inserted into the appropriate expression vector simultaneously with the polynucleotide sequence of interest (e.g., a native start codon). In such cases, additional translational control signals are not required. However, in cases where only a polypeptide coding sequence, or a portion thereof, is inserted, exogenous translational control signals, including an ATG initiation codon is provided for expression of the chimeric FG sequence. The initiation codon is placed in the correct reading frame to ensure translation of the polynucleotide sequence of interest. Exogenous transcriptional elements and initiation codons can be of various origins, both natural and synthetic.

If desired, the efficiency of expression can be further increased by the inclusion of enhancers appropriate to the cell system in use (Scharf et al. (1994) Results Probl Cell Differ 20:125-62; Bitter et al. (1987) Methods in Enzymol 153:516-544). In some instances, the nucleic acid (such as a vector) that encodes the FG polypeptide includes one or more additional sequence elements selected to increase and/or optimize expression of the FG encoding nucleic acid when introduced into a host cell. One class of expression-enhancing sequences includes an epigenetic element such as a Matrix Attachment Region (or MAR), or a similar epigenetic element, e.g., STAR elements (for example, such as those STAR elements disclosed in Otte et al., Biotechnol. Prog. 23:801-807, 2007) . Without being bound by theory, MARs are believed to mediate the anchorage of a target DNA sequence to the nuclear matrix, generating chromatin loop domains that extend outwards from the heterochromatin cores. While MARs do not contain any obvious consensus or recognizable sequence, their most consistent feature appears to be an overall high A/T content, and C bases predominating on one strand. These regions appear to form bent secondary structures that may be prone to strand separation, and may include a core-unwinding element (CUE) that can serve as the nucleation point for strand separation. Several simple AT-rich sequence motifs have been associated with MAR sequences: e.g., the A-box (AATAAAYAAA), the T-box (TTWTWTTWTT), DNA unwinding motifs (AATATATT, AATATT), SATB1 binding sites (H-box, A/T/C25) and consensus Topoisomerase II sites for vertebrates (RNYNNCNNGYNGKTNYNY) or Drosophila (GTNWAYATTNATNNR). Exemplary MAR sequences are described in published US patent application no. 20070178469, and in international patent application no. WO02/074969 (which are incorporated herein by reference). Additional MAR sequences that can be used to enhance expression of a nucleic acid encoding an FG polypeptide include chicken lysozyme MAR, MARp1-42, MARp1-6, MARp1-68, and MARpx-29, described in Girod et al., Nature Methods, 4:747-753, 2007 (disclosed in GenBank Accession Nos. EA423306, DI107030, DI106196, DI107561, and DI106512, respectively). One of skill will appreciate that expression can further be modulated be selecting a MAR that produces an intermediate level of enhancement, as is reported for MAR 1-9. If desired, alternative MAR sequences for increasing expression of a FG polypeptide can be identified by searching sequence databases, for example, using software such as MAR-Finder (available on the web at futuresoft.org/MarFinder), SMARTest (available on the web at genomatix.de), or SMARScan I (Levitsky et al., Bioinformatics 15:582-592, 1999). In certain embodiments, the MAR is introduced (e.g., transfected) into the host cell on the same nucleic acid (e.g., vector) as the FG polypeptide-encoding sequence. In an alternative embodiment, the MAR is introduced on a separate nucleic acid (e.g., in trans) and it can optionally cointegrate with the FG nucleic acid.

Exemplary procedures sufficient to guide one of ordinary skill in the art through the production of recombinant FG nucleic acids can be found in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Exemplary nucleic acids that encode chimeric FG polypeptides are represented by SEQ ID NOs: 10 and 12. Additional variants of can be produced by assembling analogous F2, F1 and G protein polypeptide sequences selected from any of the known (or subsequently) discovered strains of RSV, e.g., as shown in FIGS. 4 and 5. Additional sequence variants that share sequence identity with the exemplary variants can be produced by those of skill in the art. Typically, the nucleic acid variants will encode polypeptides that differ by no more than 1%, or 2%, or 5%, or 10%, or 15%, or 20% of the nucleotide or amino acid residues. That is, the encoded polypeptides share at least 80%, or 85%, more commonly, at least about 90% or more, such as 95%, or even 98% or 99% sequence identity. It will be immediately understood by those of skill in the art, that the polynucleotide sequences encoding the FG polypeptides, can themselves share less sequence identity due to the redundancy of the genetic code.

It will be understood by those of skill in the art, that the similarity between chimeric FG polypeptide and polynucleotide sequences, as for polypeptide and nucleotide sequences in general, can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid (or polynucleotide) sequences, the more similar are the higher order structures resulting from folding and assembly. Variants of a chimeric FG polypeptide and polynucleotide sequences can have one or a small number of amino acid deletions, additions or substitutions but will nonetheless share a very high percentage of their amino acid, and generally their polynucleotide sequence.

Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993 and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid. It will, therefore, be understood that the various variants of nucleic acids that are encompassed by this disclosure are able to hybridize to at least on of SEQ ID NOs: 5, 7, 9, 11, 13, 15, 17, 19, 67 or 69, over substantially their entire length.

### Methods of Producing Chimeric RSV Antigenic Polypeptides

The chimeric FG polypeptides disclosed herein are produced using well established procedures for the expression and purification of recombinant proteins. Procedures sufficient to guide one of skill in the art can be found in, for example, Sambrook and the Ausubel references cited above. Additional and specific details are provided hereinbelow.

Recombinant nucleic acids that encode any of the chimeric FG RSV antigens described above, such as (but not limited to) the exemplary nucleic acids represented by SEQ ID NOs:10 and 12, are introduced into host cells by any of a variety of well-known procedures, such as electroporation, liposome mediated transfection, Calcium phosphate precipitation, infection, transfection and the like, depending on the selection of vectors and host cells.

Host cells that include recombinant chimeric FG polypeptide-encoding nucleic acids are, thus, also a feature of this disclosure. Favorable host cells include prokaryotic (i.e., bacterial) host cells, such as E. coli, as well as numerous eukaryotic host cells, including fungal (e.g., yeast, such as Saccharomyces cerevisiae and Picchia pastoris) cells, insect cells, plant cells, and mammalian cells (such as CHO cells). Recombinant FG nucleic acids are introduced (e.g., transduced, transformed or transfected) into host cells, for example, via a vector, such as an expression vector. As described above, the vector is most typically a plasmid, but such vectors can also be, for example, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as 3T3, COS, CHO, BHK, HEK 293 or Bowes melanoma; plant cells, including algae cells, etc.

The host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high level expression of fusion proteins that are readily purified are favorably employed. Such vectors include, but are not limited to, multifunctional E. coli cloning and expression vectors such as BLUESCRIPT (Stratagene), in which the coding sequence of interest, e.g., a polynucleotide of the invention as described above, can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.), in which the amino-terminal methionine is ligated in frame with a histidine tag; and the like.

Similarly, in yeast, such as Saccharomyces cerevisiae, a number of vectors containing constitutive or inducible promoters such as alpha factor, alcohol oxidase and PGH can be used for production of the desired expression products. For reviews, see Berger, Ausubel, and, e.g., Grant et al. (1987; Methods in Enzymology 153:516-544). In mammalian host cells, a number expression systems, including both plasmis and viral-based systems, can be utilized.

A host cell is optionally chosen for its ability to modulate the expression of the inserted sequences or to process the expressed protein in the desired fashion. Such modifications of the protein include, but are not limited to, glycosylation, (as well as, e.g., acetylation, carboxylation, phosphorylation, lipidation and acylation). Post-translational processing for example, which cleaves a precursor form into a mature form of the protein (for example, by a furin protease) is optionally performed in the context of the host cell. Different host cells such as 3T3, COS, CHO, HeLa, BHK, MDCK, 293, WI38, etc. have specific cellular machinery and characteristic mechanisms for such post-translational activities and can be chosen to ensure the correct modification and processing of the introduced, foreign protein.

For long-term, high-yield production of recombinant chimeric FG polypeptide encoded by the nucleic acids disclosed herein, stable expression systems are typically used. For example, cell lines which stably express a chimeric FG polypeptide are introduced into the host cell using expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells which successfully express the introduced sequences. For example, resistant groups or colonies of stably transformed cells can be proliferated using tissue culture techniques appropriate to the cell type. Host cells transformed with a nucleic acid encoding a chimeric FG polypeptide are optionally cultured under conditions suitable for the expression and recovery of the encoded protein from cell culture.

Following transduction of a suitable host cell line and growth of the host cells to an appropriate cell density, the selected promoter is induced by appropriate means (e.g., temperature shift or chemical induction) and cells are cultured for an additional period. The secreted polypeptide product is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed chimeric FG polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

In certain examples, the nucleic acids are introduced into vectors suitable for introduction and expression in prokaryotic cells, e.g., E. coli cells. For example, a nucleic acid including a polynucleotide sequence that encodes a FG chimeric RSV antigen can be introduced into any of a variety of commercially available or proprietary vectors, such as the pET series of expression vectors (e.g., pET19b and pET21d). Expression of the coding sequence is inducible by IPTG, resulting in high levels of protein expression. The polynucleotide sequence encoding the chimeric RSV antigen is transcribed under the phage T7 promoter. Alternate vectors, such as pURV22 that include a heat-inducible lambda pL promoter are also suitable.

The expression vector is introduced (e.g., by electroporation) into a suitable bacterial host. Numerous suitable strains of E. coli are available and can be selected by one of skill in the art (for example, the Rosetta and BL21 (DE3) strains have proven favorable for expression of recombinant vectors containing polynucleotide sequences that encode FG chimeric RSV antigens.

In another example, the polynculeotides that encode the chimeric FG polypeptides are cloned into a vector sutiable for introduction into mammalian cells (e.g., CHO cells). In this exemplary embodiment, the polynucleotide sequence that encodes the chimeric RSV antigen is introduced into the the pEE14 vector developped by Lonza Biologicals firm. The chimeric polypeptide is expressed under a constitutive promoter, the immediate early CMV (CytoMegaloVirus) promoter. Selection of the stably transfected cells expressing the chimer is made based on the ability of the transfected cells to grow in the absence of a glutamine source. Cells that have successfully integrated the pEE14 are able to grow in the absence of exogenous glutamine, because the pEE14 vector expresses the GS (Glutamine Synthetase) enzyme. Selected cells can be clonally expanded and characterized for expression of the chimeric polypeptide.

In another example, the polynucleotide sequence that encodes the FG chimeric RSV antigen is introduced into insect cells using a Baculovirus Expression Vector System (BEVS). Recombinant baculovirus capable of infecting insect cells can be generated using commercially available vectors, kits and/or systems, such as the BD BaculoGold system from BD BioScience. Briefly, the polynucleotide sequence encoding a FG chimeric RSV antigen is inserted into the pAcSG2 transfer vector. Then, host cells SF9 (Spodoptera frugiperda) are co-transfected by pAcSG2-chimer plasmid and BD BaculoGold, containing the linearized genomic DNA of the baculovirus Autographa californica nuclear polyhedrosis virus (AcNPV). Following transfection, homologous recombination occurs between the pACSG2 plasmid and the Baculovirus genome to generate the recombinant virus. In one example, the chimeric RSV antigen is expressed under the regulatory control of the polyhedrin promoter (pH). Similar transfer vectors can be produced using other promoters, such as the basic (Ba) and p10 promoters. Similarly, alternative insect cells can be employed, such as SF21 which is closely related to the Sf9, and the High Five (Hi5) cell line derived from a cabbage looper, Trichoplusia ni.

Following transfection and induction of expression (according to the selected promoter and/or enhancers or other regulatory elements), the expressed chimeric polypeptides are recovered (e.g., purified or enriched) and renatured to ensure folding into an antigenically active conformation. Typically, the antigenically active conformation is a multimer of chimeric FG polypeptides. Favorable, the mutlimer is a trimer.

### Immunogenuic Compositions and Methods

Also provided are immunogenic compositions including a chimeric FG polypeptides and a pharmaceutically acceptable diluent, carrier or excipient. Numerous pharmaceutically acceptable diluents and carriers and/or pharmaceutically acceptable excipients are known in the art and are described, e.g., in Remington's Pharmaceutical Sciences, by E. W. Martin, Mack Publishing Co., Easton, Pa., 15th Edition (1975).

In general, the nature of the diluent, carrier and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid carriers can be used, including for example, pharmaceutical grades of trehalose, mannitol, lactose, starch or magnesium stearate.

Accordingly, suitable excipients and carriers can be selected by those of skill in the art to produce a formulation suitable for delivery to a subject by a selected route of administration.

Particular examples are given above in Table 1. Additional excipients include, without limitation: glycerol, polyethylene glycol (PEG), glass forming polyols (such as, sorbitol, trehalose) N-lauroylsarcosine (e.g., sodium salt), L-proline, non detergent sulfobetaine, guanidine hydrochloride, urea, trimethylamine oxide, KCl, Ca²⁺, Mg²⁺, Mn²⁺, Zn²⁺ (and other divalent cation related salts), dithiothreitol (DTT), dithioerytro1,13-mercaptoethanol, Detergents (including, e.g., Tween80, Tween20, Triton X-100, NP-40, Empigen BB, Octylglucoside, Lauroyl maltoside, Zwittergent 3-08, Zwittergent 3-10, Zwittergent 3-12, Zwittergent 3-14, Zwittergent 3-16, CHAPS, sodium deoxycholate, sodium dodecyl sulphate, and cetyltrimethylammonium bromide.

In certain favorable examples, the immunogenic composition also includes an adjuvant. Suitable adjuvants for use in immunogenic compositions containing chimeric FG polypeptides are adjuvants that in combination with the FG antigens disclosed herein are safe and minimally reactogenic when administered to a subject.

One suitable adjuvant for use in combination with FG chimeric antigens is a non-toxic bacterial lipopolysaccharide derivative. An example of a suitable non-toxic derivative of lipid A, is monophosphoryl lipid A or more particularly 3-Deacylated monophoshoryl lipid A (3D-MPL). 3D-MPL is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

Said lipopolysaccharide, such as 3D-MPL, can be used at amounts between 1 and 50 μg, per human dose of the immunogenic composition. Such 3D-MPL can be used at a level of about 25 μg, for example between 20-30 μg, suitably between 21-29 μg or between 22 and 28 μg or between 23 and 27 μg or between 24 and 26 μg, or 25 μg. In another embodiment, the human dose of the immunogenic composition comprises 3D-MPL at a level of about 10 μg, for example between 5 and 15 μg, suitably between 6 and 14 μg, for example between 7 and 13 μg or between 8 and 12 μg or between 9 and 11 μg, or 10 μg. In a further embodiment, the human dose of the immunogenic composition comprises 3D-MPL at a level of about 5 μg, for example between 1 and 9 μg, or between 2 and 8 μg or suitably between 3 and 7 μg or 4 and μg, or 5 μg.

In other embodiments, the lipopolysaccharide can be β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. Nonetheless, each of these references is incorporated herein by reference. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are described as TLR-4 agonists, and include, but are not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3-[4(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462)

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which can be used are alkyl Glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signaling response through TLR-4 (Sabroe et al, JI 2003 p1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, and muramyl dipeptide (MDP). In one embodiment the TLR agonist is HSP 60, 70 or 90. Other suitable TLR-4 ligands are as described in WO 2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. For example, one suitable TLR-4 ligand is ER804057.

Additional TLR agonists are also useful as adjuvants. The term “TLR agonist” refers to an agent that is capable of causing a signaling response through a TLR signaling pathway, either as a direct ligand or indirectly through generation of endogenous or exogenous ligand. Such natural or synthetic TLR agonists can be used as alternative or additional adjuvants. A brief review of the role of TLRs as adjuvant receptors is provided in Kaisho & Akira, Biochimica et Biophysica Acta 1589:1-13, 2002. These potential adjuvants include, but are not limited to agonists for TLR2, TLR3, TLR7, TLR8 and TLR9. Accordingly, in one embodiment, the adjuvant and immunogenic composition further comprises an adjuvant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In one embodiment of the present invention, a TLR agonist is used that is capable of causing a signaling response through TLR-1. Suitably, the TLR agonist capable of causing a signaling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorfei.

In an alternative embodiment, a TLR agonist is used that is capable of causing a signaling response through TLR-2. Suitably, the TLR agonist capable of causing a signaling response through TLR-2 is one or more of a lipoprotein, a peptidoglycan, a bacterial lipopeptide from M tuberculosis, B burgdorferi or T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, a TLR agonist is used that is capable of causing a signaling response through TLR-3. Suitably, the TLR agonist capable of causing a signaling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signaling response through TLR-5. Suitably, the TLR agonist capable of causing a signaling response through TLR-5 is bacterial flagellin.

In an alternative embodiment, a TLR agonist is used that is capable of causing a signaling response through TLR-6. Suitably, the TLR agonist capable of causing a signaling response through TLR-6 is mycobacterial lipoprotein, di-acylated LP, and phenol-soluble modulin. Additional TLR6 agonists are described in WO 2003/043572.

In an alternative embodiment, a TLR agonist is used that is capable of causing a signaling response through TLR-7. Suitably, the TLR agonist capable of causing a signaling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO 2002/085905.

In an alternative embodiment, a TLR agonist is used that is capable of causing a signaling response through TLR-8. Suitably, the TLR agonist capable of causing a signaling response through TLR-8 is a single stranded RNA (ssRNA), an imidazoquinoline molecule with anti-viral activity, for example resiquimod (R848); resiquimod is also capable of recognition by TLR-7. Other TLR-8 agonists which can be used include those described in WO 2004/071459.

In an alternative embodiment, a TLR agonist is used that is capable of causing a signaling response through TLR-9. In one embodiment, the TLR agonist capable of causing a signaling response through TLR-9 is HSP90. Alternatively, the TLR agonist capable of causing a signaling response through TLR-9 is bacterial or viral DNA, DNA containing unmethylated CpG nucleotides, in particular sequence contexts known as CpG motifs. CpG-containing oligonucleotides induce a predominantly Th1 response. Such oligonucleotides are well known and are described, for example, in WO 96/02555, WO 99/33488 and U.S. Pat. Nos. 6,008,200 and 5,856,462. Suitably, CpG nucleotides are CpG oligonucleotides. Suitable oligonucleotides for use in the immunogenic compositions of the present invention are CpG containing oligonucleotides, optionally containing two or more dinucleotide CpG motifs separated by at least three, suitably at least six or more nucleotides. A CpG motif is a Cytosine nucleotide followed by a Guanine nucleotide. The CpG oligonucleotides of the present invention are typically deoxynucleotides. In a specific embodiment the internucleotide in the oligonucleotide is phosphorodithioate, or suitably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. Nos. 5,666,153, 5,278,302 and WO 95/26204.

Other adjuvants that can be used in immunogenic compositions with a chimeric FG polypeptide, e.g., on their own or in combination with 3D-MPL, or another adjuvant described herein, are saponins, such as QS21.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1, which are incorporated herein by reference. Other saponins which have been used in systemic vaccination studies include those derived from other plant species such as Gypsophila and Saponaria (Bomford et al., Vaccine, 10(9):572-577, 1992). QS21 is an Hplc purified non-toxic fraction derived from the bark of Quillaja Saponaria Molina. A method for producing QS21 is disclosed in U.S. Pat. No. 5,057,540. Non-reactogenic adjuvant formulations containing QS21 are described in WO 96/33739. The aforementioned references are incorporated by reference herein. Said immunologically active saponin, such as QS21, can be used in amounts of between 1 and 50 μg, per human dose of the immunogenic composition. Advantageously QS21 is used at a level of about 25 μg, for example between 20-30 μg, suitably between 21-29 μg or between 22-28 μg or between 23-27 μg or between 24-26 μg, or 25 μg. In another embodiment, the human dose of the immunogenic composition comprises QS21 at a level of about 10 μg, for example between 5 and 15 μg, suitably between 6-14 μg, for example between 7-13 μg or 8-12 μg or between 9-11 μg, or 10 μg. In a further embodiment, the human dose of the immunogenic composition comprises QS21 at a level of about 5 μg, for example between 1-9 μg, or between 2-8 μg or suitably between 3-7 μg or 4-6 μg, or 5 μg. Such formulations Q21 and cholesterol have been shown to be successful Th1 stimulating adjuvants when formulated together with an antigen. Thus, for example, chimeric FG polypeptides can favorably be employed in immunogenic compositions with an adjuvant comprising a combination of QS21 and cholesterol.

Optionally, the adjuvant can also include mineral salts such as an aluminium or calcium salts, in particular aluminium hydroxide, aluminium phosphate and calcium phosphate. For example, an adjuvant containing 3D-MPL in combination with an aluminium salt (e.g., aluminium hydroxide or “alum”) is suitable for formulation in an immunogenic composition containing a chimeric FG polypeptide for administration to a human subject.

Another class of suitable Th1 biasing adjuvants for use in formulations with chimeric FG polypeptides include OMP-based immunostimulatory compositions. OMP-based immunostimulatory compositions are particularly suitable as mucosal adjuvants, e.g., for intranasal administration. OMP-based immunostimulatory compositions are a genus of preparations of outer membrane proteins (OMPs, including some porins) from Gram-negative bacteria, such as, but not limited to, Neisseria species (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543), which are useful as a carrier or in compositions for immunogens, such as bacterial or viral antigens. Some OMP-based immunostimulatory compositions can be referred to as “Proteosomes,” which are hydrophobic and safe for human use. Proteosomes have the capability to auto-assemble into vesicle or vesicle-like OMP clusters of about 20 nm to about 800 nm, and to noncovalently incorporate, coordinate, associate (e.g., electrostatically or hydrophobically), or otherwise cooperate with protein antigens (Ags), particularly antigens that have a hydrophobic moiety. Any preparation method that results in the outer membrane protein component in vesicular or vesicle-like form, including multi-molecular membranous structures or molten globular-like OMP compositions of one or more OMPs, is included within the definition of Proteosome. Proteosomes can be prepared, for example, as described in the art (see, e.g., U.S. Pat. No. 5,726,292 or U.S. Pat. No. 5,985,284). Proteosomes cam also contain an endogenous lipopolysaccharide or lipooligosaccharide (LPS or LOS, respectively) originating from the bacteria used to produce the OMP porins (e.g., Neisseria species), which generally will be less than 2% of the total OMP preparation.

Proteosomes are composed primarily of chemically extracted outer membrane proteins (OMPs) from Neisseria menigitidis (mostly porins A and B as well as class 4 OMP), maintained in solution by detergent (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206). Proteosomes can be formulated with a variety of antigens such as purified or recombinant proteins derived from viral sources, including the chimeric FG polypeptides disclosed herein, e.g., by diafiltration or traditional dialysis processes. The gradual removal of detergent allows the formation of particulate hydrophobic complexes of approximately 100-200 nm in diameter (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206).

“Proteosome: LPS or Protollin” as used herein refers to preparations of proteosomes admixed, e.g., by the exogenous addition, with at least one kind of lipo-polysaccharide to provide an OMP-LPS composition (which can function as an immunostimulatory composition). Thus, the OMP-LPS composition can be comprised of two of the basic components of Protollin, which include (1) an outer membrane protein preparation of Proteosomes (e.g., Projuvant) prepared from Gram-negative bacteria, such as Neisseria meningitidis, and (2) a preparation of one or more liposaccharides. A lipo-oligosaccharide can be endogenous (e.g., naturally contained with the OMP Proteosome preparation), can be admixed or combined with an OMP preparation from an exogenously prepared lipo-oligosaccharide (e.g., prepared from a different culture or microorganism than the OMP preparation), or can be a combination thereof. Such exogenously added LPS can be from the same Gram-negative bacterium from which the OMP preparation was made or from a different Gram-negative bacterium. Protollin should also be understood to optionally include lipids, glycolipids, glycoproteins, small molecules, or the like, and combinations thereof. The Protollin can be prepared, for example, as described in U.S. Patent Application Publication No. 2003/0044425.

Combinations of different adjuvants, such as those mentioned hereinabove, can also be used in compositions with chimeric FG polypeptides. For example, as already noted, QS21 can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL: QS21. Another combination adjuvant formulation includes 3D-MPL and an aluminium salt, such as aluminium hydroxide. When formulated in combination, this combination can enhance an antigen-specific Th1 immune response.

In some instances, the adjuvant formulation includes liposomes, an oil-in-water emulsion, or a mineral salt such as a calcium or aluminium salt, for example calcium phosphate, aluminium phosphate or aluminium hydroxide.

One example of an oil-in-water emulsion comprises a metabolisable oil, such as squalene, a tocol such as alpha-tocopherol, and a surfactant, such as polysorbate 80 or Tween 80, in an aqueous carrier, and does not contain any additional immunostimulants(s), in particular it does not contain a non-toxic lipid A derivative (such as 3D-MPL) or a saponin (such as QS21). The aqueous carrier can be, for example, phosphate buffered saline. Additionally the oil-in-water emulsion can contain span 85 and/or lecithin and/or tricaprylin.

In another embodiment of the invention there is provided a vaccine composition comprising an antigen or antigen composition and an adjuvant composition comprising an oil-in-water emulsion and optionally one or more further immunostimulants, wherein said oil-in-water emulsion comprises 0.5-10 mg metabolisable oil (suitably squalene), 0.5-11 mg tocol (suitably alpha-tocopherol) and 0.4-4 mg emulsifying agent.

In one specific embodiment, the adjuvant formulation includes 3D-MPL prepared in the form of an emulsion, such as an oil-in-water emulsion. In some cases, the emulsion has a small particle size of less than 0.2 μm in diameter, as disclosed in WO 94/21292. For example, the particles of 3D-MPL can be small enough to be sterile filtered through a 0.22 micron membrane (as described in European Patent number 0 689 454). Alternatively, the 3D-MPL can be prepared in a liposomal formulation. Optionally, the adjuvant containing 3D-MPL (or a derivative thereof) also includes an additional immunostimulatory component.

For example, when an immunogenic composition with a chimeric FG polypeptide antigen is formulated for administration to an infant, the dosage of adjuvant is determined to be effective and relatively non-reactogenic in an infant subject. Generally, the dosage of adjuvant in an infant formulation is lower than that used in formulations designed for administration to adult (e.g., adults aged 65 or older). For example, the amount of 3D-MPL is typically in the range of 1 μg-200 μg, such as 10-100 μg, or 10 μg-50 μg per dose. An infant dose is typically at the lower end of this range, e.g., from about 1 μg to about 50 μg, such as from about 2 μg, or about 5 μg, or about 10 μg, to about 25 μg, or to about 50 μg. Typically, where QS21 is used in the formulation, the ranges are comparable (and according to the ratios indicated above). For adult and elderly populations, the formulations typically include more of an adjuvant component than is typically found in an infant formulation. In particular formulations using an oil-in-water emulsion, such an emulsion can include additional components, for example, such as cholesterol, squalene, alpha tocopherol, and/or a detergent, such as tween 80 or span85. In exemplary formulations, such components can be present in the following amounts: from about 1-50 mg cholesterol, from 2 to 10% squalene, from 2 to 10% alpha tocopherol and from 0.3 to 3% tween 80. Typically, the ratio of squalene: alpha tocopherol is equal to or less than 1 as this provides a more stable emulsion. In some cases, the formulation can also contain a stabilizer. Where alum is present, e.g., in combination with 3D-MPL, the amount is typically between about 100 μg and 1 mg, such as from about 100 μg, or about 200 μg to about 750 μg, such as about 500 μg per dose.

An immunogenic composition typically contains an immunoprotective quantity (or a fractional dose thereof) of the antigen and can be prepared by conventional techniques. Preparation of immunogenic compositions, including those for administration to human subjects, is generally described in Pharmaceutical Biotechnology, Vol. 61 Vaccine Design-the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. 4,372,945 and by Armor et al., U.S. Pat. 4,474,757.

Typically, the amount of protein in each dose of the immunogenic composition is selected as an amount which induces an immunoprotective response without significant, adverse side effects in the typical subject. Immunoprotective in this context does not necessarily mean completely protective against infection; it means protection against symptoms or disease, especially severe disease associated with the virus. The amount of antigen can vary depending upon which specific immunogen is employed. Generally, it is expected that each human dose will comprise 1 1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg. The amount utilized in an immunogenic composition is selected based on the subject population (e.g., infant or elderly). An optimal amount for a particular composition can be ascertained by standard studies involving observation of antibody titres and other responses in subjects. Following an initial vaccination, subjects can receive a boost in about 4 weeks.

### EXAMPLES

### Example 1

### Exemplary Chimeric RSV Polypeptide Antigens

Exemplary Eukaryotic FG Polypeptides.

Exemplary eukaryotic chimeric FG V1-1 and FG V2-1 were produced in accordance with this disclosure. The sequence of such exemplary FG chimeras are provided in SEQ ID NOs:10 and 11. The chimeric FG polypeptides included the F0 native signal sequence. Incorporation of a signal sequence enhances post-translational modifications, such as glycosylation. In these exemplary embodiments, both furin recognition motifs were removed, and a linker was inserted between the F2 and F1 domains. The sequence of the linkers present in FG V1-1 and FG V2-1 are provided in SEQ ID NOs:5 and 6, respectively.

This exemplary recombinant protein was designed to be expressed in mammalian Chinese Hamster Ovary (CHO) cells using a GS expression system. CHO cells grown in glutamine-free medium require exogenous glutamine for optimal growth. Following transfection of CHO cells with a pEE14 vector including a polynucleotide sequence encoding a chimeric FG polypeptide, this system enables selection of stable clones via metabolic deprivation, due to expression of glutamine synthase by the pEE14 vector. Although the constructs described here were produced for expression in CHO cells, these constructs can equally be produced for expression using a Baculovirus Expression Vector System (BEVS).

### Example 2

### Neutralization Inhibition in Human Sera by Chimeric RSV Polypeptide

Human sera obtained from volunteers were screened for reactivity against RSV A by ELISA and used in the neutralization inhibition (NI) assay at relevant dilution based on prior RSV neutralization potential titration. Sera were mixed with inhibitor proteins at concentrations of 25 μg/ml and incubated 1.5 to 2 hours at 37° C. In a round bottom 96-well plate, sera and proteins were mixed with a fixed concentration of RSV A and incubated for 20 min at 33° C. The sera-inhibitor-virus mixtures was then placed into previously Vero cell-seeded flat bottom 96-well plates, and further incubated for 5-6 days at 33° C. with 5% CO2 until immunofluorescence assay for NI titer detection.

Titers were calculated using the Reed-Muench method and percentages of NI calculated as follow: [(NI titer of 25 μg/ml inhibitor−NI titer of 0 μg/ml inhibitor)/NI titer of 0 μg/ml inhibitor]×100.

| Sequence Listing | |
|---|---|
| Nucleotide sequence encoding RSV reference Fusion protein | |
| Strain A2 GenBank Accession No. U50362 | |
| SEQ ID NO: 1 | |
| atggagttgctaatcctcaaagcaaatgcaattaccacaatcctcactgcagtcacatttgttttgcttctggtcaa | |
| aacatcactgaagaattttatcaatcaacatgcagtgcagtagcaaaggctatcttagtgctctgagaactggttgg | |
| tataccagtgttataactatagattaagtaatatcaaggaaaataagtgtaatggaacagatgctaaggtaaaattg | |
| ataaacaagaattagataaatataaaaatgctgtaacagaattgcagttgctcatgcaaagcacccagcaacaaaca | |
| atcgagccagaagagaactaccaaggtttatgaattatacactcaaaatgccaaaaaaaccaatgtaacattaagca | |
| agaaaaggaaaagaagatttcttggtttttgttaggtgttggatctgcaatcgccagtggcgttgctgtatctaagg | |
| tcctgcacctgaaggggaagtgaacaagatcaaaagtgctctactatccacaaacaaggctgtagtcagttatcaaa | |
| tggagttagtgtcttaaccagcaaagtgttagacctcaaaaactatatagaaaacaattgttacctattgtgaacaa | |
| gcaaagctgcagcatatcaaatatagcaactgtatagagttccaacaaaagaacaacagactactagagattaccag | |
| ggaatttagtgttaagcaggtgtaactacacctgtaagcacttacatgttaactaatagtgaattattgtcattatc | |
| aatgatatgcctataacaaatgatcagaaaaagttaatgtccaacaatgttcaaatgttagacagcaaagttactct | |
| atcatgtccataataaaagaggaagtcttagcatatgtgtacaattaccactatatggtgttatagatacaccctgt | |
| tggaaactacacacatccccctatgtacaaccaacacaaaagaagggtccaacatctgtttaacaagaactgacaga | |
| ggtggtactgtgacaatgcaggatcagtatctttcttcccacaagctgaaacatgtaaagtcaatcaaatcgagtat | |
| tttgtgacacaatgaacagtttaacattaccaagtgaagtaaactctgcaatgttgacatattcaaccccaaatatg | |
| attgtaaaattatgacttcaaaaacgatgtaagcagctccgttatcacatctctaggagccattgtgtcatgctatg | |
| gcaaaacaaatgtacagcatccaataaaaatcgtggaatcataaagacattttctaacgggtgcgatatgtatcaaa | |
| taaaggggtggacactgtgtctgtaggtaacacattatattatgtaaaaagcaagaaggtaaaagtctctatgtaaa | |
| aggtgaaccaataataaatttctatgacccttagtattcccctctgatgaatttgatgcatcaatatctcaagtcaa | |
| cgagaagattaacagagcctagcatttattcgtaaatccgatgaattattacataatgtaaatgctggtaatccacc | |
| ataaatatcatgataactactataattatagtgattatagtaatattgttatcttaattgctgttggactgctctta | |
| tactgtaaggccagaagcacaccagtcacactaagaaagatcaactgagtggtataaataatattgcatttagtaac | |
| taa | |
| Amino acid sequence of RSV reference F protein precursor F0 | |
| Strain A2 GenBank Accession No. AAB86664 | |
| SEQ ID NO: 2 | |
| MELLILKANAITTILTAVTFCFASGQNITEEFYQSTCSAVSKGYLSALRTGWYTSVITIELSNIKENKCNGTDAKVK | |
| LIKQELDKYKNAVTELQLLMQSTPATNNRARRELPRFMNYTLNNAKKTNVTLSKKRKRRFLGFLLGVGSAIASGVAV | |
| SKVLHLEGEVNKIKSALLSTNKAVVSLSNGVSVLTSKVLDLKNYIDKQLLPIVNKQSCSISNIATVIEFQQKNNRLL | |
| EITREFSVNAGVTTPVSTYMLTNSELLSLINDMPITNDQKKLMSNNVQIVRQQSYSIMSIIKEEVLAYVVQLPLYGV | |
| IDTPCWKLHTSPLCTTNTKEGSNICLTRTDRGWYCDNAGSVSFFPQAETCKVQSNRVFCDTMNSLTLPSEVNLCNVD | |
| IFNPKYDCKIMTSKTDVSSSVITSLGAIVSCYGKTKCTASNKNRGIIKTFSNGCDYVSNKGVDTVSVGNTLYYVNKQ | |
| EGKSLYVKGEPIINFYDPLVFPSDEFDASISQVNEKINQSLAFIRKSDELLHNVNAGKSTINIMITTIIIVIIVILL | |
| SLIAVGLLLYCKARSTPVTLSKDQLSGINNIAFSN | |
| Nucleotide sequence encoding RSV reference G protein | |
| Strain Long | |
| SEQ ID NO: 3 | |
| Atgtccaaaaacaaggaccaacgcaccgctaagacactagaaaagacctgggacactctcaatcatttattattcat | |
| atcatcgggcttatataagttaaatcttaaatctatagcacaaatcacattatccattctggcaatgataatctcaa | |
| cttcacttataattacagccatcatattcatagcctcggcaaaccacaaagtcacactaacaactgcaatcatacaa | |
| gatgcaacaagccagatcaagaacacaaccccaacatacctcactcaggatcctcagcttggaatcagcttctccaa | |
| tctgtctgaaattacatcacaaaccaccaccatactagcttcaacaacaccaggagtcaagtcaaacctgcaaccca | |
| caacagtcaagactaaaaacacaacaacaacccaaacacaacccagcaagcccactacaaaacaacgccaaaacaaa | |
| ccaccaaacaaacccaataatgattttcacttcgaagtgtttaactttgtaccctgcagcatatgcagcaacaatcc | |
| aacctgctgggctatctgcaaaagaataccaaacaaaaaaccaggaaagaaaaccaccaccaagcctacaaaaaaac | |
| caaccttcaagacaaccaaaaaagatctcaaacctcaaaccactaaaccaaaggaagtacccaccaccaagcccaca | |
| gaagagccaaccatcaacaccaccaaaacaaacatcacaactacactgctcaccaacaacaccacaggaaatccaaa | |
| actcacaagtcaaatggaaaccttccactcaacctcctccgaaggcaatctaagcccttctcaagtctccacaacat | |
| ccgagcacccatcacaaccctcatctccacccaacacaacacgccagtag | |
| Amino acid sequence of RSV reference G protein | |
| SEQ ID NO: 4 | |
| MSKNKDQRTAKTLEKTWDTLNHLLFISSGLYKLNLKSIAQITLSILAMIISTSLIITAIIFIASANHKVTLTTAIIQ | |
| DATSQIKNTTPTYLTQDPQLGISFSNLSEITSQTTTILASTTPGVKSNLQPTTVKTKNTTTTQTQPSKPTTKQRQNK | |
| PPNKPNNDFHFEVFNFVPCSICSNNPTCWAICKRIPNKKPGKKTTTKPTKKPTFKTTKKDLKPQTTKPKEVPTTKPT | |
| EEPTINTTKTNITTTLLTNNTTGNPKLTSQMETFHSTSSEGNLSPSQVSTTSEHPSQPSSPPNTTRQ | |
| Synthetic Linker 1 | |
| SEQ ID NO: 5 | |
| SGGSGGSGGSGGSG | |
| Synthetic Linker 2 | |
| SEQ ID NO: 6 | |
| SGGSGGSGTNVTLS | |
| Synthetic Linker 3 | |
| SEQ ID NO: 7 | |
| ELPRFMNYTLNNTKNTNVTLS | |
| Synthetic Linker 4 | |
| SEQ ID NO: 8 | |
| QYTLNNTKNTNVTLS | |
| FG-Rixensart Original(WO | |
| SEQ ID NO: 9 | |
| 1 | atggagttgc caatcctcaa agcaaatgca attaccacaa tcctcgctgc agtcacattt |
| 61 | tgctttgctt ctagtcaaaa catcactgaa gaattttatc aatcaacatg cagtgcagtt |
| 121 | agcaaaggct atcttagtgc tctaagaact ggttggtata ctagtgttat aactatagaa |
| 181 | ttaagtaata tcaaggaaaa taagtgtaat ggaacagatg ctaaggtaaa attgatgaaa |
| 241 | caagaattag ataaatataa aaatgctgta acagaattgc agttgctcat gcaaagcaca |
| 301 | ccagcagcaa acaatcgagc cagaagagaa ctaccaaggt ttatgaatta tacactcaac |
| 361 | aataccaaaa aaaccaatgt aacattaagc aagaaaagga aaagaagatt tcttggtttt |
| 421 | ttgttaggtg ttggatctgc aatcgccagt ggcattgctg tatctaaggt cctgcactta |
| 481 | gaaggagaag tgaacaagat caaaagtgct ctactatcca caaacaaggc cgtagtcagc |
| 541 | ttatcaaatg gagttagtgt cttaaccagc aaagtgttag acctcaaaaa ctatatagat |
| 601 | aaacaattgt tacctattgt gaataagcaa agctgcagaa tatcaaatat agaaactgtg |
| 661 | atagagttcc aacaaaagaa caacagacta ctagagatta ccagggaatt tagtgttaat |
| 721 | gcaggtgtaa ctacacctgt aagcacttac atgttaacta atagtgaatt attgtcatta |
| 781 | atcaatgata tgcctataac aaatgatcag aaaaagttaa tgtccaacaa tgttcaaata |
| 841 | gttagacagc aaagttactc tatcatgtcc ataataaaag aggaagtctt agcatatgta |
| 901 | gtacaattac cactatatgg tgtgatagat acaccttgtt ggaaattaca cacatcccct |
| 961 | ctatgtacaa ccaacacaaa agaagggtca aacatctgtt taacaagaac tgacagagga |
| 1021 | tggtactgtg acaatgcagg atcagtatct ttcttcccac aagctgaaac atgtaaagtt |
| 1081 | caatcgaatc gagtattttg tgacacaatg aacagtttaa cattaccaag tgaagtaaat |
| 1141 | ctctgcaatg ttgacatatt caatcccaaa tatgattgta aaattatgac ttcaaaaaca |
| 1201 | gatgtaagca gctccgttat cacatctcta ggagccattg tgtcatgcta tggcaaaact |
| 1261 | aaatgtacag catccaataa aaatcgtgga atcataaaga cattttctaa cgggtgtgat |
| 1321 | tatgtatcaa ataaaggggt ggacactgtg tctgtaggta acacattata ttatgtaaat |
| 1381 | aagcaagaag gcaaaagtct ctatgtaaaa ggtgaaccaa taataaattt ctatgaccca |
| 1441 | ttagtattcc cctctgatga atttgatgca tcaatatctc aagtcaatga gaagattaac |
| 1501 | cagagtttag catttattcg taaatccgat gaattattac ataatgtaaa tgctggtaaa |
| 1561 | tcaaccacaa atatcctggt cacactaaca actgcaatca tacaagatgc aacaagccag |
| 1621 | atcaagaaca caaccccaac atacctcacc cagaatcccc agcttggaat cagcttctcc |
| 1681 | aatctgtctg aaactacatc acaaaccacc accatactag cttcaacaac accaagtgtc |
| 1741 | aagtcaaccc tgcaatccac aacagtcaag accaaaaaca caacaacaac caaaatacaa |
| 1801 | cccagcaagc ccaccacaaa acaacgccaa aacaaaccac caaacaaacccaataatgat |
| 1861 | tttcactttg aagtgttcaa ctttgtacct tgcagcatat gcagcaacaatccaacctgc |
| 1921 | tgggctatct gtaaaagaat accaaacaaa aaacctggaa agaaaaccac caccaagccc |
| 1981 | acaaaaaaac caaccatcaa gacaaccaaa aaagatctca aacctcaaac cacaaaacca |
| 2041 | aaggaagtac ctaccaccaa gcccacagaa aagccaacca tcaacaccac caaaacaaac |
| 2101 | atcagaacta cactgctcac caacaatacc acaggaaatc cagaacacac aagtcaaaag |
| 2161 | ggaaccctcc actcaacctc ctccgatggc aatccaagcc cttcacaagt ctatacaaca |
| 2221 | tccgagtacc tatcacaacc tccatctcca tccaacacaa caaaccag |
| FG V1-1 (CHO) | |
| SEQ ID NO: 10 | |
| 1 | aagcttgcca ccatggagct gctgatcctg aaaaccaacg ccatcaccgc catcctggcc |
| 61 | gccgtgaccc tgtgcttcgc ctcctcccag aacatcaccg aagagtttta ccagtccacc |
| 121 | tgctccgccg tgtccaaggg ctacctgtcc gccctgcgga ccggctggta cacctccgtg |
| 181 | atcaccatcg agctgtccaa catcaaagaa aacaagtgca acggcaccga cgccaaggtc |
| 241 | aagctgatca agcaggaact ggacaagtac aagagcgccg tgacagaact ccagctcctg |
| 301 | atgcagtcca cccctgccac caacaacaag aagtccggcg gcagcggcgg ctctggcggc |
| 361 | tccggcggat ctggcaagaa gttcctgggc ttcctgctgg gcgctggctc cgccatcgcc |
| 421 | tccggcaccg ccgtgagcaa ggtgctgcac ctggagggcg aggtgaacaa gatcaagagc |
| 481 | gccctgctgt ccaccaacaa ggccgtggtg tccctgtcca acggcgtgtc cgtgctgacc |
| 541 | tccaaggtgc tggatctgaa gaactacatc gacaagcagc tgctgcctat cgtgaacaag |
| 601 | cagtcctgct ccatctccaa catcgagacc gtgatcgagt tccagcagaa gaacaaccgg |
| 661 | ctgctggaga tcacccgcga gttctccgtg aacgccggcg tgaccacccc tgtgtccacc |
| 721 | tacatgctga ccaactccga gctgctgtcc ctgatcaacg acatgcctat caccaacgac |
| 781 | caaaaaaagc tgatgtccaa caacgtgcag atcgtgcggc agcagtccta cagcatcatg |
| 841 | agcatcatca aggaagaagt cctggcctac gtcgtgcagc tgcctctgta cggcgtgatc |
| 901 | gacacccctt gctggaagct gcacacctcc cccctgtgca ccaccaacac caaggaaggc |
| 961 | tccaacatct gcctgacccg gaccgaccgg ggctggtact gcgacaacgc cggctccgtg |
| 1021 | tccttcttcc ctctggccga gacctgcaag gtgcagtcca accgggtgtt ctgcgacacc |
| 1081 | atgaactccc tgaccctgcc ttccgaggtg aacctgtgca acatcgacat cttcaacccc |
| 1141 | aagtacgact gcaagatcat gaccagcaag accgacgtgt cctccagcgt gatcacctcc |
| 1201 | ctgggcgcca tcgtgtcctg ctacggcaag accaagtgca ccgcctccaa caagaaccgg |
| 1261 | ggaatcatca agaccttctc caacggctgc gactacgtgt ccaataaggg cgtggacacc |
| 1321 | gtgtccgtgg gcaacacact gtactacgtg aataagcagg aaggcaagag cctgtacgtg |
| 1381 | aagggcgagc ctatcatcaa cttctacgac cctctggtgt tcccttccga cgagttcgac |
| 1441 | gcctccatca gccaggtcaa cgagaagatc aaccagtccc tggccttcat ccggaagtcc |
| 1501 | gacgagctgc tgcacaacgt gaacgctggc aagtctacca ccaacatcat ggtgaccaag |
| 1561 | cagcggcaga acaagcctcc taacaagccc aacaacgact tccacttcga ggtgttcaac |
| 1621 | ttcgtgcctt gctccatctg ctccaacaac cctacctgct gggccatctg caagagaatc |
| 1681 | cccaacaaga agccaggcaa gaaaaccacc accaagccta ccaagaagcc taccttcaag |
| 1741 | accaccaaga aggaccacaa gcctcagacc acaaagccta aggaagtgcc aaccaccaag |
| 1801 | caccaccacc atcaccactg ataatcta |
| FG V1-1 | |
| SEQ ID NO: 11 | |
| MELLILKTNAITAILAAVTLCFASSQNITEEFYQSTCSAVSKGYLSALRTGWYTSVITIELSNIKENKCNGTDAKVK | |
| LIKQELDKYKSAVTELQLLMQSTPATNNKKSGGSGGSGGSGGSGKKFLGFLLGAGSAIASGTAVSKVLHLEGEVNKI | |
| KSALLSTNKAVVSLSNGVSVLTSKVLDLKNYIDKQLLPIVNKQSCSISNIETVIEFQQKNNRLLEITREFSVNAGVT | |
| TPVSTYMLTNSELLSLINDMPITNDQKKLMSNNVQIVRQQSYSIMSIIKEEVLAYVVQLPLYGVIDTPCWKLHTSPL | |
| CTTNTKEGSNICLTRTDRGWYCDNAGSVSFFPLAETCKVQSNRVFCDTMNSLTLPSEVNLCNIDIFNPKYDCKIMTS | |
| KTDVSSSVITSLGAIVSCYGKTKCTASNKNRGIIKTFSNGCDYVSNKGVDTVSVGNTLYYVNKQEGKSLYVKGEPII | |
| NFYDPLVFPSDEFDASISQVNEKINQSLAFIRKSDELLHNVNAGKSTTNIMVTKQRQNKPPNKPNNDFHFEVFNFVP | |
| CSICSNNPTCWAICKRIPNKKPGKKTTTKPTKKPTFKTTKKDHKPQTTKPKEVPTTKHHHHHH | |
| FG V2-1 | |
| SEQ ID NO: 12 | |
| 1 | aagcttgcca ccatggagct gctgatcctc aagaccaacg ccatcaccgc catcctggcc |
| 61 | gccgtgaccc tgtgcttcgc ctcctcccag aacatcaccg aagagttcta ccagtccacc |
| 121 | tgctccgccg tgtccaaggg ctacctgtcc gccctgcgga ccggctggta cacctccgtg |
| 181 | atcaccatcg agctgtccaa catcaaagaa aacaagtgca acggcaccga cgccaaggtc |
| 241 | aagctgatca agcaggaact ggacaagtac aagagcgccg tgaccgaact ccagctgctg |
| 301 | atgcagtcca cccctgccac caacaacaag aaagaactgc ctcggttcat gaactacacc |
| 361 | ctgaacaaca ccaagaacac caacgtgacc ctgagcaaga agttcctggg cttcctgctg |
| 421 | ggcgctggct ccgccatcgc ctccggcacc gccgtgagca aggtgctgca cctggagggc |
| 481 | gaggtgaaca agatcaagag cgccctgctg tccaccaaca aggccgtggt gtccctgtcc |
| 541 | aacggcgtgt ccgtgctgac ctccaaggtg ctggatctga agaactacat cgacaagcag |
| 601 | ctgctgccta tcgtgaacaa gcagtcctgc tccatctcca acatcgagac cgtgatcgag |
| 661 | ttccagcaga agaacaaccg gctgctggag atcacccgcg agttctccgt gaacgccggc |
| 721 | gtgaccaccc ctgtgtccac ctacatgctg acaaactccg agctgctctc cctgatcaac |
| 781 | gacatgccta tcaccaacga ccaaaaaaag ctgatgtcca acaacgtgca gatcgtgcgg |
| 841 | cagcagtcct acagcatcat gagcatcatc aaggaagagg tcctggccta cgtggtgcag |
| 901 | ctgcctctgt acggcgtgat cgacacccct tgctggaagc tgcacacctc ccccctgtgc |
| 961 | accaccaaca ccaaggaagg ctccaacatc tgcctgaccc ggaccgaccg gggctggtac |
| 1021 | tgcgacaacg ccggctccgt gtccttcttc cctctggccg agacctgcaa ggtgcagtcc |
| 1081 | aaccgggtgt tctgcgacac catgaactcc ctgaccctgc cttccgaggt gaacctgtgc |
| 1141 | aacatcgaca tcttcaaccc caagtacgac tgcaagatca tgaccagcaa gaccgacgtg |
| 1201 | tcctccagcg tgatcacctc cctgggcgcc atcgtgtcct gctacggcaa gaccaagtgc |
| 1261 | accgcctcca acaagaaccg gggaatcatc aagaccttct ccaacggctg cgactacgtg |
| 1321 | tccaataagg gcgtggacac cgtgtccgtg ggcaacacac tgtactacgt gaataagcag |
| 1381 | gaaggcaaga gcctgtacgt gaagggcgag cctatcatca acttctacga ccctctggtg |
| 1441 | ttcccttccg acgagttcga cgcctccatc agccaggtca acgagaagat caaccagtcc |
| 1501 | ctggccttca tccggaagtc cgacgagctg ctgcacaacg tgaacgctgg caagtctacc |
| 1561 | accaacatca tggtgaccaa gcagcggcag aacaagcctc ctaacaagcc caacaacgac |
| 1621 | ttccacttcg aggtgttcaa cttcgtgcct tgctccatct gctccaacaa ccctacctgc |
| 1681 | tgggccatct gcaagagaat ccccaacaag aagcctggca agaaaaccac caccaagcct |
| 1741 | accaagaagc ctaccttcaa gaccaccaag aaggaccaca agcctcagac cacaaagcct |
| 1801 | aaggaagtgc caaccaccaa gcaccaccac catcaccact gataatcta |

FG V2-1
SEQ ID NO: 13
MELLILKTNAITAILAAVTLCFASSQNITEEFYQSTCSAVSKGYLSALRTGWYTSVITIELSNIKENKCNGTDAKVK
LIKQELDKYKSAVTELQLLMQSTPATNNKKELPRFMNYTLNNTKNTNVTLSKKFLGFLLGAGSAIASGTAVSKVLHL
EGEVNKIKSALLSTNKAVVSLSNGVSVLTSKVLDLKNYIDKQLLPIVNKQSCSISNIETVIEFQQKNNRLLEITREF
SVNAGVTTPVSTYMLTNSELLSLINDMPITNDQKKLMSNNVQIVRQQSYSIMSIIKEEVLAYVVQLPLYGVIDTPCW
KLHTSPLCTTNTKEGSNICLTRTDRGWYCDNAGSVSFFPLAETCKVQSNRVFCDTMNSLTLPSEVNLCNIDIFNPKY
DCKIMTSKTDVSSSVITSLGAIVSCYGKTKCTASNKNRGIIKTFSNGCDYVSNKGVDTVSVGNTLYYVNKQEGKSLY
VKGEPIINFYDPLVFPSDEFDASISQVNEKINQSLAFIRKSDELLHNVNAGKSTTNIMVTKQRQNKPPNKPNNDFHF
EVFNFVPCSICSNNPTCWAICKRIPNKKPGKKTTTKPTKKPTFKTTKKDHKPQTTKPKEVPTTKHHHHHH

1. A chimeric RSV polypeptide comprising in an N terminal to C terminal direction:
(i) a first amino acid sequence comprising an F2 domain uncleavably joined to an F1 domain of a Respiratory Syncytial Virus (RSV) Fusion (F) protein polypeptide; and
(ii) a second amino acid sequence comprising a portion of an RSV Attachment (G) protein polypeptide comprising an immunologically dominant epitope.

2. The chimeric RSV polypeptide of claim 1, wherein the F2 domain and the F1 domain of the RSV F protein polypeptide are uncleavably joined via an amino acid linker.

3-5. (canceled)

6. The chimeric RSV polypeptide of claim 1, further comprising a signal peptide.

7. The chimeric RSV polypeptide of claim 1, wherein the F2 domain comprises an amino acid sequence from residue 24 to residue 105 of a native F protein polypeptide.

8. The chimeric RSV polypeptide of claim 1, wherein the F1 domain comprises an amino acid sequence from residue 137 to residue 528 of a native F protein polypeptide.

9. The chimeric RSV polypeptide of claim 1, wherein the portion of the RSV G protein polypeptide comprises from amino acid residue 183 to residue 203 of a native G protein polypeptide.

10. The chimeric RSV polypeptide of claim 1, wherein the portion of the RSV G protein polypeptide comprises from amino acid residue 152 to residue 229 of a native G protein polypeptide.

11. The chimeric RSV polypeptide of claim 1, wherein the portion of the RSV G protein polypeptide comprises from amino acid residue 149 to residue 229 of a native G protein polypeptide.

12.-15. (canceled)

16. The chimeric RSV polypeptide of claim 1, wherein the chimeric polypeptide comprises an amino acid sequence selected from SEQ ID NOs:11 and 13 or a subsequence thereof.

17-18. (canceled)

19. A recombinant RSV antigen comprising a multimer of the chimeric RSV polypeptide of claim 1.

20. (canceled)

21. An immunogenic composition comprising the chimeric RSV polypeptide of claim 1, and a carrier or excipient.

22-24. (canceled)

25. The immunogenic composition of claim 21, further comprising an adjuvant.

26-36. (canceled)

37. The immunogenic composition of claim 21, wherein the immunogenic composition reduces or prevents infection with RSV, or reduces or prevents a pathological response caused by said infection, following administration to a human subject.

38. (canceled)

39. The immunogenic composition of any of claim 21, further comprising at least one additional antigen of a pathogenic organism other than RSV.

40-42. (canceled)

43. A recombinant nucleic acid comprising a polynucleotide sequence that encodes the chimeric polypeptide of claim 1.

44-46. (canceled)

47. A host cell comprising the nucleic acid of claim 0.

48-50. (canceled)

51. A method for eliciting an immune response against Respiratory Syncytial Virus (RSV), the method comprising the step of:
administering to a subject an immunogenically effective amount of a composition comprising the chimeric RSV polypeptide of claim 1.

52. The method of claim 51, wherein administering the composition comprising the chimeric RSV polypeptide elicits an immune response specific for RSV without enhancing viral disease following contact with RSV.

53. The method of claim 52, wherein the immune response comprises a Th1-type immune response.

54. The method of claim 52, wherein the immune response comprises a protective immune response that reduces or prevents infection with a RSV and/or reduces or prevents a pathological response following infection with a RSV.

55-57. (canceled)
